(19) United States
(12) Patent Application Publication
(10) Pub. No.: US 2025/0157613 A1
(43) Pub. Date: May 15, 2025

(54) COMMUNICATION TAG FOR A DRUG PRODUCT

(71) Applicant: Eli Lilly and Company, Indianapolis, IN (US)

(72) Inventors: Douglas James GORR, Indianapolis, IN (US); Senthil Subramanian RAJARATNAM, Carmel, IN (US); Louis Stevens SOMLAI, Zionsville, IN (US)

(21) Appl. No.: 18/835,368

(22) PCT Filed: Jan. 27, 2023
(86) PCT No.: PCT/US2023/011668

§ 371 Date: Aug. 2, 2024

Related U.S. Application Data
(60) Provisional application No. 63/307,874, filed on Feb. 8, 2022.

(51) Int. Cl. G16H 20/13; A61M 5/31; A61M 5/315; G06F 21/44; G16H 20/17

(52) U.S. Cl. CPC G16H 20/13; A61M 5/31; G06F 21/44; G16H 20/17; A61M 2005/3125; A61M 5/3158; A61M 2205/3368; A61M 2205/3592

(57) ABSTRACT

A medication delivery device is provided having a housing comprising a reservoir sized sufficiently to hold medication, an actuating button for initiating an injection of the medication, and a label affixed on at least a portion of the housing. The label comprises a communication tag configured to, when accessed by a mobile device, cause the mobile device to launch a web browser configured to access a uniform resource locator (URL). The mobile device is configured to receive data from a communication tag disposed on the medication delivery device and verify and/or authenticate an aspect of the medication delivery device or track possession of the medication delivery device based on the received data.

### BACKGROUND

Counterfeiters sell products that are packaged to look as though they were manufactured by a certain manufacturer, when, in fact, they were not. Consuming counterfeit medications can be unsafe and may prevent a patient from receiving the medication that is needed to treat his or her disease or condition. As a result, such counterfeit products can pose concerns for both patients and manufacturers.

### SUMMARY

According to an exemplary embodiment of the present disclosure, a medication delivery device is provided. The medication delivery device includes a housing including a reservoir sized sufficiently to hold medication. The medication delivery device includes an actuating button for initiating an injection of the medication. The medication delivery device includes a label affixed on at least a portion of the housing. The label includes a communication tag configured to, when accessed by a mobile device, cause the mobile device to launch a web browser configured to access a uniform resource locator (URL). In some embodiments, the communication tag may be a RFID tag. In some embodiments, the communication tag may be a Near Field Communication (NFC) tag.

According to another embodiment of the present disclosure, a computerized method for verifying an aspect of a medication delivery device is provided. The method includes receiving data from a communication tag disposed on the medication delivery device. The medication delivery device includes a housing including a reservoir sized sufficiently to hold medication. The medication delivery device includes an actuating button for initiating an injection of the medication. The medication delivery device includes a label affixed on at least a portion of the housing. The label includes the tag. The method includes verifying the aspect of the medication delivery device by transmitting at least a portion of the received data to a server.

According to yet another embodiment of the present disclosure, a computerized method for tracking possession of a medication delivery device is provided. The method includes receiving data from a communication tag disposed on the medication delivery device. The medication delivery device includes a housing including a reservoir sized sufficiently to hold medication. The medication delivery device includes an actuating button for initiating an injection of the medication. The medication delivery device includes a label affixed on at least a portion of the housing. The label includes the tag. The method includes transmitting at least a portion of the received data to a server. The method includes providing user credentials to the server. The server is configured to log an entry based on the received data and the user credentials, the entry indicating that the user was in possession of the medication delivery device at a time that the data was received from the tag disposed on the medication delivery device.

### BRIEF DESCRIPTION OF THE DRAWINGS

Additional embodiments of the disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 is a block diagram depicting an exemplary system 100 for verifying the aspect of a medication delivery device, according to some embodiments.

FIG. 2 is a flowchart showing an exemplary computerized method 200 for verifying an aspect of a medication delivery device, according to some embodiments.

FIG. 3 is a block diagram depicting exemplary information displayed on a user interface of a mobile device, according to some embodiments.

FIG. 4A is an example display on a user interface of a mobile device showing verification data associated with the medication delivery device, according to some embodiments.

FIG. 4B is an example display on a user interface of a mobile device showing verification data associated with the medication delivery device, according to some embodiments.

FIG. 4C is an example display on a user interface of a mobile device showing a recall notice associated with the medication contained within the medication delivery device, according to some embodiments.

FIG. 4D is an example display on a user interface of a mobile device showing information indicating an expiration of a medication contained within the medication delivery device, according to some embodiments.

FIG. 5 is a block diagram depicting an example system 500 for tracking possession of a medication delivery device, according to some embodiments.

FIG. 6 is a flowchart showing an exemplary computerized method 600 for tracking possession of a medication delivery device, according to some embodiments.

FIG. 7 is a block diagram depicting an example system 700 for using a logged entry to determine example recommendations associated with the medication delivery device, according to some embodiments.

FIG. 8A is a block diagram depicting an example system 800a for displaying first information on the user interface based on receiving user credentials for a first user, according to some embodiments.

FIG. 8B is a block diagram depicting an example system 800b for displaying second information on the user interface based on receiving user credentials for a second user, according to some embodiments.

FIG. 9A is a block diagram depicting an example system 900 for using multiple logged entries from multiple users to determine an average level of adherence, according to some embodiments.

FIG. 9B is a block diagram depicting an example system 950 for using multiple logged entries from a single user to determine an average level of adherence, according to some embodiments.

FIG. 10 is a block diagram depicting an example system 1000 for tracking product distribution, according to some embodiments.

FIG. 11 is a block diagram depicting an example system 1100 for detecting a counterfeit product, according to some embodiments.

FIG. 12 is a block diagram depicting an example system for using a patient-specific medication delivery device and an example system for using a health care provider (HCP)-specific medication delivery device, according to some embodiments.

FIG. 13 is a block diagram depicting an example system for configuring dosing reminders, according to some embodiments.

FIG. 14A illustrates an exemplary medication delivery device in its initial pre-use configuration, according to some embodiments.

FIG. 14B illustrates the medication delivery device after its end cap has been removed, but before the device has been activated to deliver medication.

FIG. 14C illustrates the medication delivery device after the device has been activated to deliver medication.

FIG. 15A shows an exemplary label that includes an NFC tag, according to some embodiments.

FIG. 15B shows a view of the back of the exemplary label.

FIG. 16 shows an illustrative implementation of a computer system that may be used to perform any of the aspects of the techniques and embodiments disclosed herein, according to some embodiments.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

Provided herein are techniques for verifying an aspect of a medication delivery device. According to some embodiments, the techniques verify the aspect of the medication delivery device based on data received from a communication tag disposed on the medication delivery device. The communication tag may be a RFID tag and/or a NFC tag, or any other type of tag capable of communicating data to a reader device. For example, the data may include data indicative of an authentication code generated by the tag. According to some embodiments, the techniques transmit at least a portion of the data received from the tag to a server. For example, the portion of the data may include data indicative of the authentication code. According to some embodiments, the server is configured to authenticate the authentication code and generate verification information indicative of a result of the authenticating. For example, the verification information may indicate whether a source (e.g., a manufacturer and/or supplier) of the medication delivery device is verified and, by extension, whether the medication delivery device is a verified product, as opposed to a counterfeit product.

Also provided herein are techniques for tracking possession of a medication delivery device. According to some embodiments, the techniques track possession of the medication delivery device based on a time that data was received from a tag disposed on the medication delivery device. After receiving data from the tag, the techniques launch a website prompting a user to provide user credentials. For example, the website may prompt a user to provide a username and password, a bearer token, a refresh token (e.g., derived from previous authentication, such as via using a username and password), and/or other user credentials. According to some embodiments, the techniques provide the user credentials to a server. The server is configured to log an entry based on the user credentials and the data received from the tag. For example, the server may log an entry indicating that a user who is associated with the provided user credentials was in possession of the medication delivery device at the time that data was received from the tag disposed on the medication delivery device. According to some embodiments, the entry may also indicate a geographical location where the data was received from the tag, information about the medication contained within the medication delivery device, and/or information about the user associated with the user credentials. This geographical location may be derived from an IP address from which the server received the data from the tag, and/or by location data supplied by a mobile device (e.g., a smartphone) associated with the user.

The inventors have appreciated that product safety and liability concerns arise in drug distribution channels that are not strictly regulated. In particular, counterfeiters may sell medications that are packaged to look like they are from a certain manufacturer, but which are, in fact, counterfeit products. The inventors have appreciated that such counterfeit products can pose problems. For example, such products can pose safety concerns for end patients who need assurance that the medication they purchased was in fact a genuine drug article manufactured by the verified manufacturer. As another example, if a counterfeit product causes one or more problems to an end patient and the patient does not realize it is a counterfeit product, it may negatively affect the reputation of the true manufacturer (when, in fact, the true manufacturer had nothing to do with the problems caused by the counterfeit product).

Accordingly, the inventors have developed techniques for verifying an aspect of a medication delivery device and for tracking possession of a medication delivery device. In some embodiments, the techniques can evaluate data received from a communication tag disposed on a medication delivery device that is indicative of a unique authentication code. The authentication code changes over time in an unpredictable manner that is only known to a server configured to authenticate the authentication code. For example, the authentication code may change based on activity, such that each time a mobile device is brought within proximity to the medication delivery device to enable the mobile device to communicate with the tag, the authentication code would change. The inventors have appreciated that the use of a tag that generates such an unpredictable authentication code is useful for preventing counterfeiters from being able to successfully counterfeit at least this aspect of the medication delivery device. According to some embodiments, the techniques verify an aspect of the medication delivery device (e.g., a source of the medication delivery device) by transmitting at least some of the data received from the tag (e.g., the authentication code) to the server.

While embodiments of the technology described herein describe techniques for verifying an aspect of and tracking possession of a medication delivery device, it should be appreciated that such techniques may be applied to any suitable type of medication container. For example, the techniques described herein may be used to verify an aspect of and track possession of a pill bottle, a drug vial, a blister pack, or any other suitable type of medication container, as aspects of the technology are not limited in this respect. According to some embodiments, a tag is disposed on such a medication container and is configured to communicate with a mobile device.

In some embodiments, the communication tag may comprise any electronic circuit configured to communicate wirelessly with a reader device. For example, the communication tag may be a RFID tag and/or a NFC tag. For ease of explication, the balance of the disclosure refers to the communication tag as an NFC tag, but it should be understood that other types of wireless communication tags may also be used.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

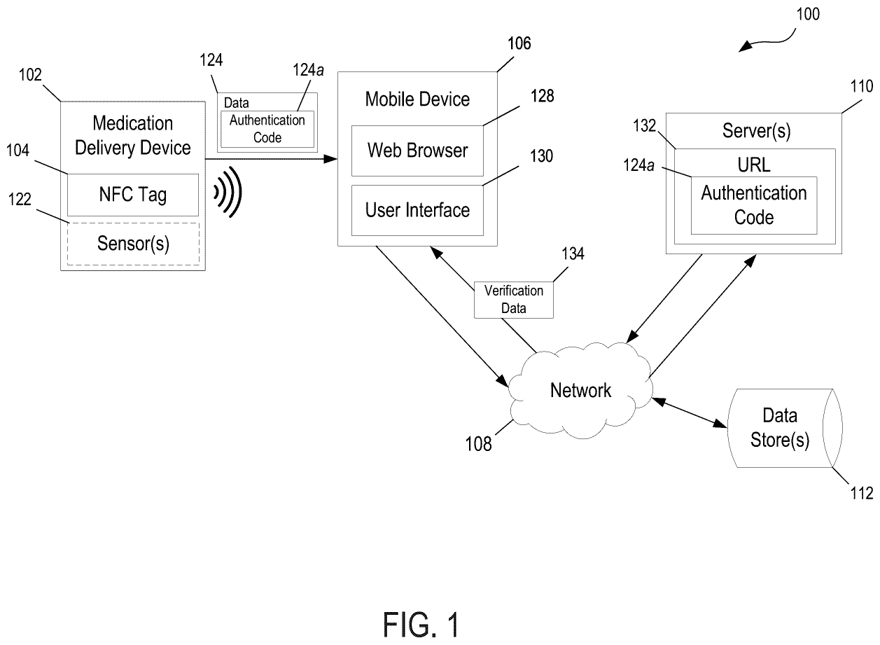

FIG. 1 is a block diagram depicting an exemplary system 100 for verifying an aspect of a medication delivery device, according to some embodiments. System 100 includes medication delivery device 102 and mobile device 106, which communicates with server(s) 110 and data store(s) 112 via network 108. It should be appreciated that system 100 is illustrative and that a system may have one or more components of any suitable type in addition to or instead of the components illustrated in FIG. 1.

In some embodiments, the mobile device 106 is configured to receive data 124 from a near field communication (NFC) tag 104 disposed on the medication delivery device 102. For example, the mobile device 106 may receive data 124 when it is in proximity to (e.g., within 10 cm, within 6 cm, within 4 cm, etc.) to the medication delivery device 102.

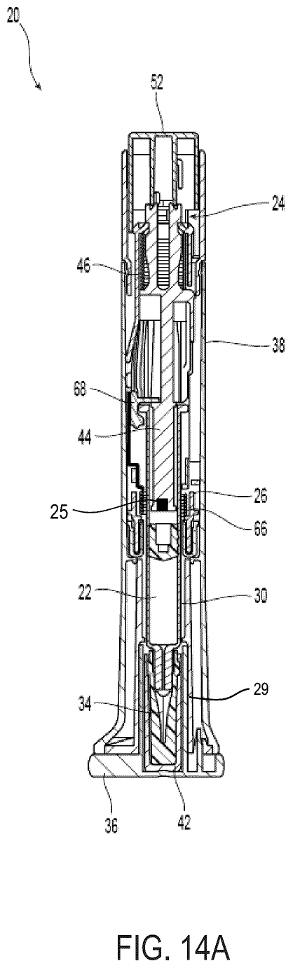

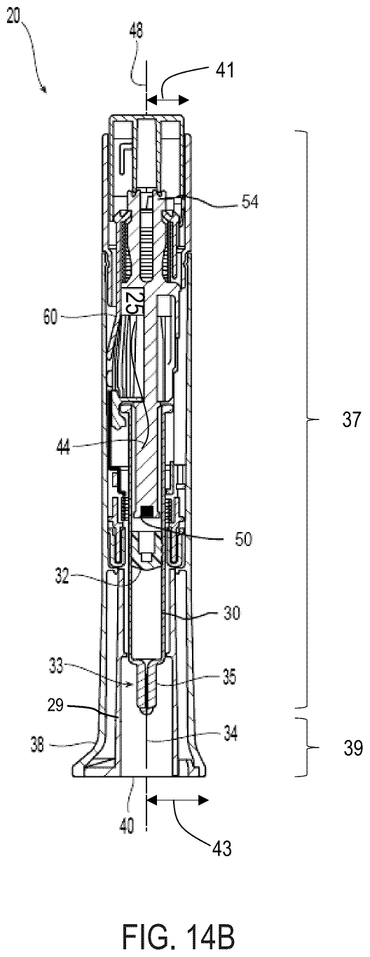

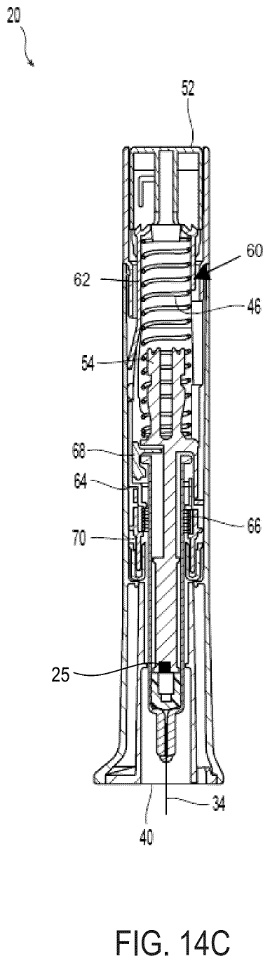

Medication delivery device 102 may include any suitable medication delivery device, such as an autoinjector or a pre-filled syringe, as aspects of the technology are not limited in this respect. An exemplary medication delivery device 20 is described herein in more detail, including at least with respect to FIGS. 14A-14C.

In some embodiments, the medication delivery device 102 optionally includes sensor(s) 122. For example, the medication delivery device 102 may include a temperature sensor configured to sense a temperature. The sensed temperature may be a temperature of the medication delivery device 102, a temperature of medication contained within the medication delivery device 102, and/or ambient temperature. The temperature sensor(s) may take the form of any suitable sensor for sensing temperature, such as but not limited to a thermistor (e.g., a negative temperature coefficient (NTC) thermistor or a resistance temperature detector (RTD)), a thermocouple, or a semiconductor-based temperature sensor.

Additionally or alternatively, the sensor(s) 122 may include sensor(s) for determining other parameters of the medication delivery device 102, such as an amount of medication remaining in the device's reservoir, a type of medication stored within the device's reservoir, an amount of medication selected for delivery, an amount of medication actually delivered, whether the medication delivery device is in contact with skin tissue, and the like.

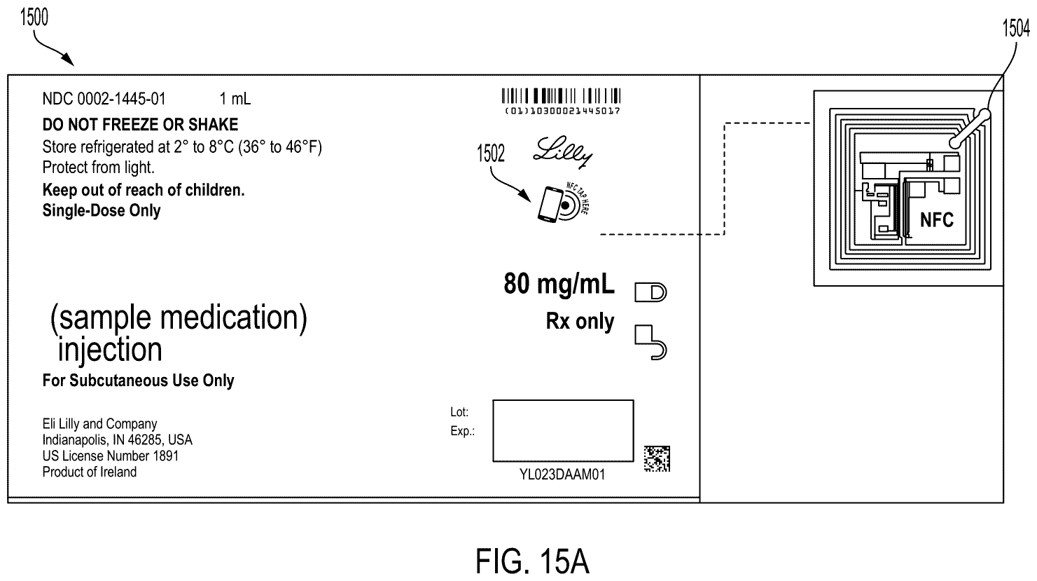

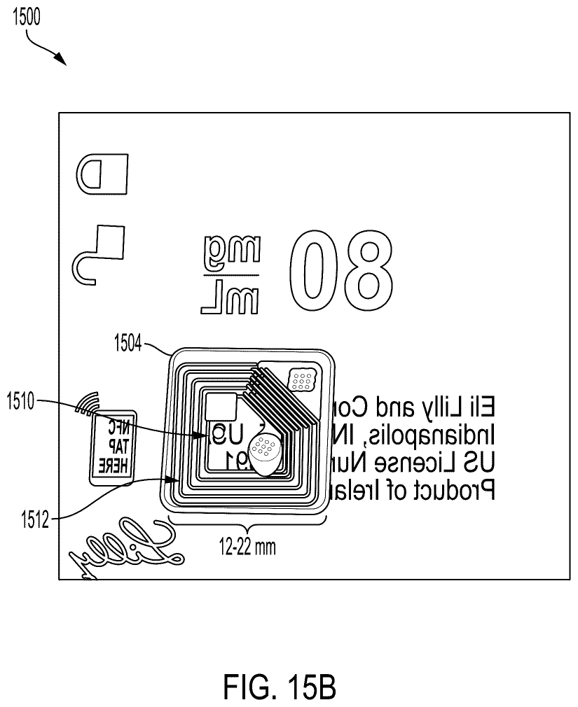

In some embodiments, an NFC tag 104 is disposed on the medication delivery device. For example, NFC tag 104 may be incorporated into a label of the medication delivery device 102. The label may be affixed to, adhered to, or formed as part of the medication delivery device 102. The label may be paper, polymer, or made of any other suitable material, as aspects of the technology described herein are not limited in this respect. An exemplary label, which incorporates an NFC tag, is described herein including at least with respect to FIGS. 15A and 15B.

In some embodiments, the NFC tag 104 includes an NFC chip coupled to NFC antenna(s). The NFC chip may take the form of any suitable NFC chip, such as but not limited to an NFC chip manufactured by NXP®. The NFC antenna(s) may take the form of any suitable NFC antenna(s), such as but not limited to the NFC antenna(s) manufactured Avery Dennison® or Identiv. However, it should be appreciated that aspects of the technology described herein are not limited to any particular NFC tag.

NFC tag 104 is configured to communicate data 124 to and/or from mobile device 106 using an NFC wireless transmission protocol. Data 124 includes data associated with the NFC tag 104, data associated with the medication delivery device 102, data associated with the medication contained within the medication delivery device 102, and/or data associated with the sensor(s) 122. Accordingly, sensor(s) 122 may be communicably coupled to NFC tag 104.

The data associated with the NFC tag 104 includes data indicative of an authentication code 124a generated by the NFC tag 104. When the mobile device 106 is placed in proximity to the NFC tag, the NFC tag generates an authentication code that is only known to server(s) 110. For example, a new authentication code may be generated each time mobile device 106 and/or any other suitable mobile device is placed in proximity to the medication delivery device 102 so as to enable such mobile device to communicate with NFC tag 104. In some embodiments, the authentication code is encrypted prior to transmission to mobile device 106.

The data associated with the sensor(s) 122 includes data indicative of the output of the sensor(s) 122. For example, when the sensor(s) 122 include a temperature sensor, the data may indicate the temperature of the medication delivery device 102, the temperature of medication contained within the medication delivery device 102, and/or the ambient temperature.

According to some embodiments, in response to receiving data 124 from the NFC chip 104, the mobile device determines information indicative of an event. An “event,” as used herein, refers to the occurrence of a mobile device being placed in proximity to the medication delivery device 102 such that data (e.g., data 124) is transmitted to the mobile device. The information indicative of the event may include contextual information such as the time, date, and/or geographical location of the mobile device 106 when it received data 124 from the medication delivery device 102. According to some embodiments, the mobile device 106 determines such contextual information based on the on-board clock, calendar, and/or GPS chip of the mobile device 106 at the time the data was received from the NFC tag 104.

Mobile device 106 may comprise any device that receives, stores, and/or processes data 124 from NFC tag 104 via a wireless signal received by a communication circuit of the mobile device 106. Exemplary mobile devices include a smartphone, a smartwatch, a tablet, and/or a laptop. The wireless signal may be an active signal, in which the mobile device 106 receives signals transmitted by a communication circuit of the medication delivery device 102, or it may be a passive signal, in which mobile device 106 senses modulations to a signal transmitted by the NFC tag 104. Mobile device 106 may include a separate communication circuit configured to communicate with other devices (e.g., using long-range or cellular transmission protocols).

Mobile device 106 may further include a processing circuit and memory (not shown). The processing circuit may take the form of a processor (e.g., a microprocessor or microcontroller, field-programmable gate arrays (FPGAs) and/or digital signal processors (DSPs, or any combination of the foregoing) configured to execute logic stored in a memory to perform the operations described herein. The term “logic”, “control logic”, “instructions” or “application” as used herein may include software and/or firmware executing on any of the aforementioned processing circuits. The memory may be any suitable computer readable medium that is accessible by the processing circuit and includes both volatile and non-volatile memory. Exemplary memory includes random-access memory (RAM), read-only memory (ROM), electrically erasable programmable ROM (EEPROM), flash memory, a magnetic storage device, optical disk storage, or any other suitable medium which is configured to store data and which is accessible by the processor circuit, whether directly or indirectly via one or more intermediary devices or wired or wireless communication links. Although the preceding description assumes that the memory is separate from but communicably coupled to the processing circuit, in some embodiments the memory may also be integrated with the processing circuit. In some embodiments, instead of a processor that executes logic stored in memory, the processing circuit may take the form of hard-wired logic, e.g., a state machine and/or an application-specific integrated circuit (ASIC) that performs the functions described herein.

In some embodiments, the mobile device 106 includes a user interface 130 for displaying data and/or receiving user input. For example, the user interface 130 may comprise a graphical user interface (GUI) including a touchscreen display. The touchscreen display allows the user to interact with presented information, menus, buttons, and other data to provide information to the user or to receive user input from the user. Additionally or alternatively, a keyboard, keypad, microphone, mouse pointer, or other suitable user input device may be provided.

Upon receiving data 124 from the medication delivery device 102, mobile device 106 launches a web browser 128 configured to access a uniform resource locator (URL) 132 associated with server(s) 110 via network 108. In some embodiments, a website associated with the URL 132 is displayed on the user interface 130 of the mobile device 106. Additionally or alternatively, upon receiving data 124 the mobile device searches for and launches a previously-downloaded application program (e.g., mobile app) configured to access the URL 132. The received data 124 may then be passed to the mobile app via server(s) 110. Additionally or alternatively, the data 124 may be directly transmitted from the medication delivery device 102 to the server(s) 110.

In some embodiments, server(s) 110 include one or multiple computing devices. When server(s) 110 include multiple computing devices, the device(s) may be physically co-located (e.g., in a single room) or distributed across multiple physical locations. In some embodiments, server(s) 110 may be part of a cloud computing infrastructure. In some embodiments, one or more server(s) 110 may be co-located in a facility operated by an entity.

Network 108 may be or include a wide area network (e.g., the Internet), a local area network (e.g., a corporate Internet), and/or any other suitable type of network. Any of the devices shown in FIG. 1 may connect to the network 108 using one or more wired links, one or more wireless links, and/or any suitable combination thereof. Accordingly, the network 108 may be, for example, a hard-wired network (e.g., a local area network within a healthcare facility), a wireless network (e.g., connected over Wi-Fi and/or cellular networks), a cloud-based computing network, or any combination thereof.

The URL 132 may be associated with the authentication code 124a provided by the NFC tag 104 disposed on the medication delivery device 102. In some embodiments, the server(s) 110 include an authentication server configured to authenticate the medication delivery device 102 based on the authentication code 124a. For example, the authentication server may authenticate the authentication code 124a to verify whether the NFC tag 104 and the associated medication delivery device 102 is a genuine product manufactured and/or provided by a verified source (e.g., a verified manufacturer, provider, etc.). In some embodiments, authentication server authenticates the authentication code 124a by decrypting the authentication code 124a (e.g., when the authentication code 124a is encrypted by NFC tag 104). The authentication server then looks up the authentication code 124a in data store(s) 112 to determine whether it is authentic.

Additionally or alternatively, server(s) 110 may query data store(s) 112 to identify additional information associated with the authentication code 124a and, by extension, the medication delivery device 102 from which the authentication code 124a was received. For example, the information may include information related to the medication contained within medication delivery device 102, such as medication name, medication dosage, recall notices, expiration date, and manufacturing lot number. The information may indicate a dose regimen and/or instructions for taking the medication. The information may include information related to one or more preceding events, such as times and/or locations of preceding events.

In some embodiments, data store(s) 112 include any suitable data store, such as a flat file, a data store, a multi-file, or data storage of any suitable type, as aspects of the technology described herein are not limited to any particular type of data store.

In some embodiments, server(s) 110 are configured to evaluate data associated with sensor(s) 122. Server(s) 110 may determine temperature-based information, such as whether the medication contained in the medication delivery device 102 is warm enough to be administered. Additionally or alternatively, server(s) 110 may determine dosage-based information, such as an amount of medication that was dispensed and/or programmed, based on the data detected by sensor(s) 122.

In some embodiments, the server(s) 110 transmit verification data 134 to mobile device 106 (e.g., to web browser 128 and/or to a mobile app). The verification data 134 may include data indicative of the authentication of the medication delivery device 102 and/or the NFC tag 104. Additionally or alternatively, the server(s) 110 transmit additional data (not shown) to mobile device 106. The additional data includes the information associated with the sensor(s) 122 and/or the information associated with the authentication code 124a and the medication delivery device 102.

In some embodiments, at least some of the verification data 134 and/or additional data may be part of a GUI (e.g., user interface 130) presented to the user. In some embodiments, the GUI may be presented to the user as part of the website displayed by the web browser 128 executing on the mobile device 106. In some embodiments, the GUI may be presented to the user using an application program (different from the web browser 128) executing on the mobile device 106.

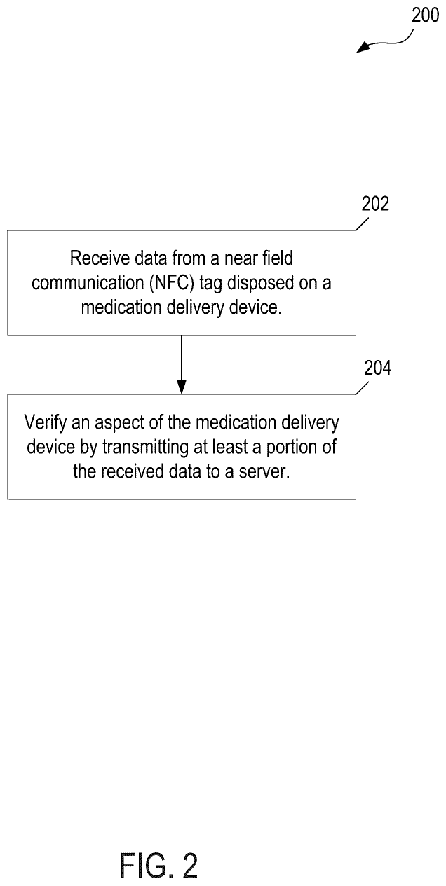

FIG. 2 is a flowchart showing an exemplary computerized method 200 for verifying an aspect of a medication delivery device, according to some embodiments. Method 200 may be implemented on any of one or more of mobile device 106 and server(s) 110, whether working independently or in cooperation with one another.

At step 202, the mobile device receives data from a near field communication (NFC) tag (e.g., NFC tag 104) disposed on a medication delivery device (e.g., medication delivery device 102). The NFC tag may include an NFC chip and one or more NFC antennas disposed on the medication delivery device. For example, the NFC tag may include an NFC chip and one or more NFC antennas incorporated into a label of the medication delivery device. The label may be affixed to, adhered to, or formed as part of the medication delivery device. An exemplary label, which incorporates an NFC tag, is described herein including at least with respect to FIGS. 15A and 15B.

In some embodiments, the mobile device receives the data in response to coming within proximity to the medication delivery device. This may include coming within 10 cm, within 8 cm, within 6 cm, within 4 cm, within 2 cm, between 0 and 10 cm, between 2 and 8 cm, or within any suitable distance of the medication delivery device. For example, a user can tap the medication delivery device against the mobile device (or vice versa), and, as a result, the mobile device receives data from the medication delivery device. The mobile device may receive the data via a wireless signal received by a communication circuit of the mobile device. The wireless signal may be an active signal or a passive signal.

The received data may include data associated with the NFC tag disposed on the medication delivery device and/or data associated with one or more sensors associated with the medication delivery device. As described herein, including at least with respect to FIG. 1, data associated with the NFC tag may include data indicative of an authentication code generated by the NFC tag. In some embodiments, the data indicative of the authentication code is encrypted data. In some embodiments, the data associated with the one or more sensors includes data indicative of the output of the one or more sensors, such as the sensors described herein including at least with respect to FIG. 1.

According to some embodiments, in response to receiving data from the NFC chip, the mobile device determines information indicative of an event. Such information may include contextual information such as the time, date, and/or geographical location of the mobile device when it received data from the medication delivery device. According to some embodiments, the mobile device determines the contextual information based on one or more components of the mobile device, such as the on-board clock, calendar, and/or GPS chip of the mobile device at the time the data was received from the NFC tag.

After receiving the data (e.g., in response to receiving the data), the mobile device launches a web browser configured to access a URL associated with a server. Additionally or alternatively, the mobile device checks for and launches a previously downloaded application program (e.g., a mobile app) configured to access the URL associated with the server.

At step 204, the mobile device verifies an aspect of the medication delivery device by transmitting at least a portion (e.g., some, most, or all) of the received data and/or the contextual information to the server. In some embodiments, the data that is transmitted to the server includes data indicative of the authentication code generated by the NFC tag.

The server may be configured to authenticate the authentication code and generate verification data based on a result of the authenticating. For example, the server may be configured to decrypt the data indicative of the authentication code (e.g., when the data is encrypted) and query a data store for the (decrypted) authentication code. If the authentication code is found in the data store, the verification data indicates that the aspect of the medication delivery device has been verified. If the authentication code is not found in the data store, the verification data indicates that the aspect of the medication delivery device cannot be verified.

In some embodiments, the authentication code may change over time in a way that is predictable to the server but relatively unpredictable to other entities (e.g., to eavesdropping attackers). For example, the code may change every time the NFC tag is brought within close proximity to a mobile device such that the mobile device may query the NFC tag. Every time the NFC tag is queried by a mobile device, the mobile device may be configured to alert the server, such that the server may keep track of how many times the NFC tag has been queried and how the authentication code is evolving over time. In such embodiments, the server may authenticate the authentication code by determining whether the authentication code matches an expected authentication code at the current time.

Verifying an aspect of the medication delivery device may include verifying a source of the medication delivery device. For example, this may include verifying that a particular manufacturer manufactured the medication delivery device and/or that a particular supplier supplied the medication delivery device. In some embodiments, if the aspect of the medication delivery device cannot be verified, this indicates that the medication delivery device was not received from a verified source (e.g., the medication delivery device is a counterfeit from an unverified manufacturer and/or supplier). If the aspect of the medication delivery device can be verified, this indicates that the medication delivery device was received from a verified source (e.g., from the correct manufacturer and/or supplier).

In some embodiments, the server is further configured to obtain additional data associated with the authentication code and, by extension, the medication delivery device. For example, the server may look up the authentication code in a data store to identify additional information about the medication delivery device and/or the medication contained within the medication delivery device. The additional information may include information such as medication name, medication dosage, recall notices, expiration date, and manufacturing lot number. The information may indicate a dose regimen and/or instructions for taking the medication. The information may include information related to one or more preceding events, such as times and/or locations of preceding events.

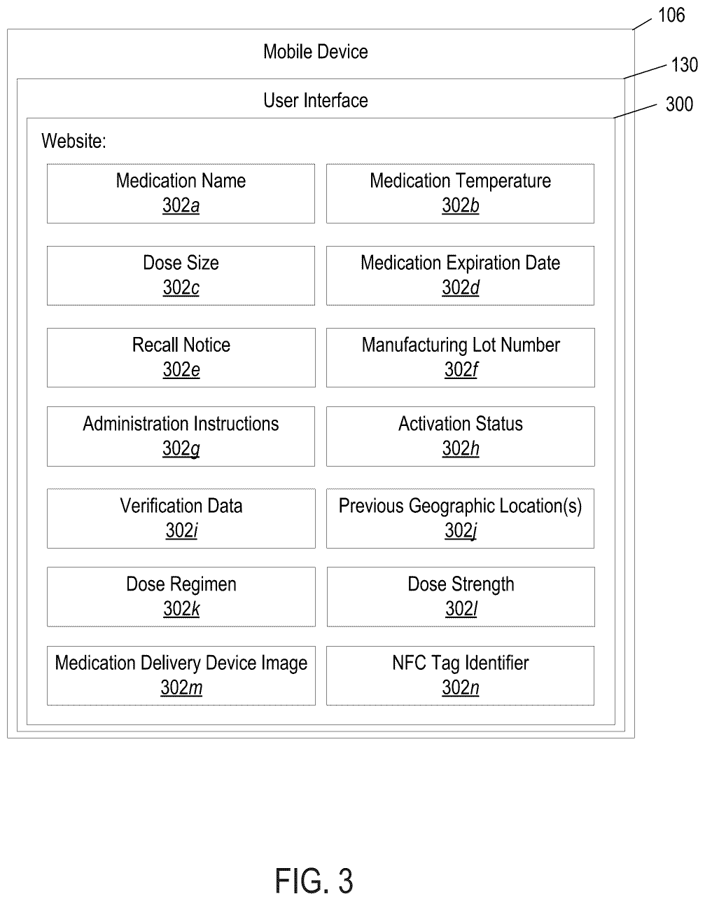

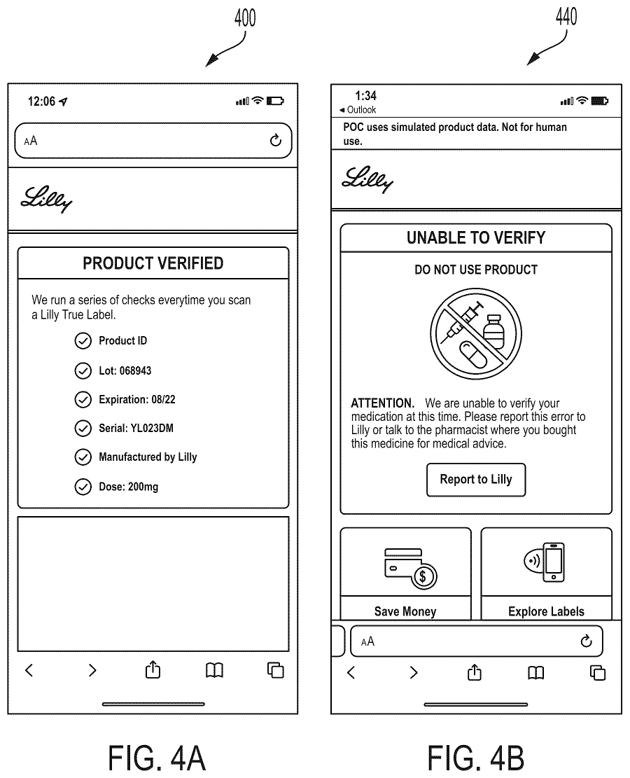

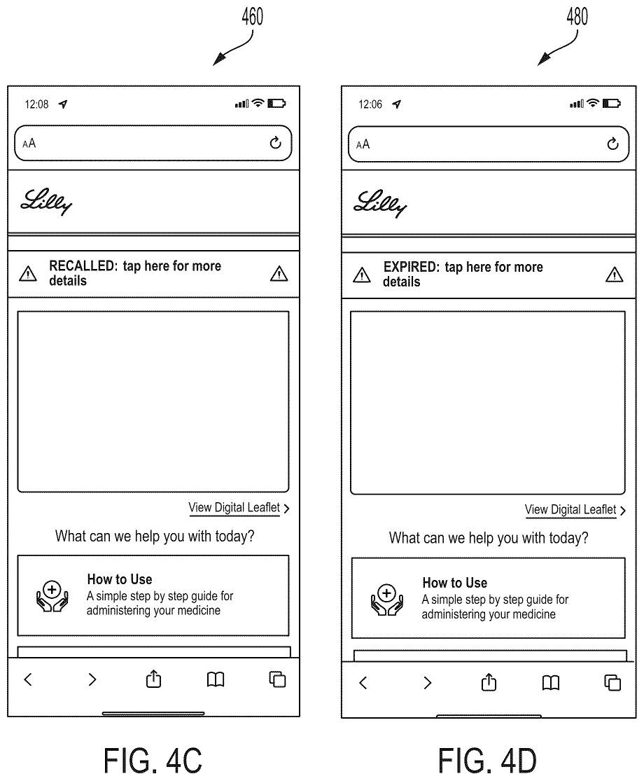

The mobile device is configured to receive the verification data and/or the additional data from the server and present at least a portion of the received data on a website displayed by the web browser or application executing on the mobile device. The mobile device may be configured to present a GUI as part of the website. Nonlimiting examples of the information that can be displayed on the mobile device are described herein including at least with respect to FIGS. 3 and 4A-4D.

FIG. 3 is a block diagram depicting exemplary information that can be displayed on the user interface 130 of the mobile device 106, according to some embodiments. As shown, the information is presented as part of a website 300 displayed by a web browser (e.g., web browser 128) of the mobile device 106. However, though not shown, it should be appreciated that the information may be presented using an application program executing on the mobile device 106.

In some embodiments, the information is distributed across one or multiple pages of the website 300 (or app). The website 300 may include a GUI, and a user may interact with the GUI to navigate the pages of the website 300. For example, the GUI may include one or more buttons, scroll bars, text boxes, or any other suitable elements with which the user can interact. In some embodiments, the information described herein may be presented using one or more audible messages, which may be helpful for users who are blind and/or visually impaired. Such audible messages may be provided in addition to, or as an alternative to, presenting such information visually.

In some embodiments, the information includes information indicative of verification data 302i. As described herein, including at least with respect to FIGS. 1 and 2, the verification data 302i may be obtained from a server (e.g., server(s) 110) and indicate whether an aspect of the medication delivery device (e.g., a source of the medication delivery device) is verified. When the aspect of the medication delivery device cannot be verified, the verification data 302i may indicate that the medication delivery device is a counterfeit. For example, a warning message may be displayed on website 300. Additionally or alternatively, when the aspect of the medication delivery device can be verified, the verification data 302i may indicate that the medication delivery device was received from a verified source. For example, a message may be displayed on website 300 indicating to the user that aspects of the medication delivery device have been verified.

In some embodiments, the displayed information includes information associated with the medication delivery device and the medication contained within the medication delivery device. For example, the information includes factual data about the medication, such as the medication name 302a, dose size 302c (e.g., a size of a dose of medication contained in medication delivery device), the expiration date 302d, a dose strength 3021 and/or the manufacturing lot number 302f In some embodiments, the information includes an image of the medication delivery device 302m. In some embodiments, if the medication and/or medication delivery device are recalled after they have been distributed to patients, the information includes a recall notice 302e. In some embodiments, the information may indicate a dose regimen 302k and/or provide administration instructions 302g for the patient or caregiver. For example, the information may indicate that the dose of medication contained in the medication delivery device is part of a multi-dose regimen and indicate the order in which the doses should be taken. Administration instructions may include step-by-step instructions for administering the medication using the medication delivery device. Such instructions are useful to a patient or caregiver (e.g., a family member, a nurse, a first responder, etc.) who is administering the medication.

In some embodiments, the information includes information related to the output of one or more sensors associated with the medication delivery device. For example, the information includes the medication temperature 302b obtained as output from one or more temperature sensors associated with the medication delivery device. In some embodiments, the information may include temperature-based information, such as whether the medication contained in the medication delivery device is warm enough to be administered. Additionally or alternatively, the information includes an activation status 302h obtained as output from a sensor configured to determine an activation state of the medication delivery device. For example, the activation status may indicate whether the medication delivery device has been activated to dispense medication or whether the medication delivery device has dispensed medication. In some embodiments, the information may include dosage-based information (e.g., based on the activation status of the medication delivery device) that indicates an amount of medication that has been dispensed and/or programmed to be dispensed.

In some embodiments, the information includes information related to the NFC tag 104 disposed on the medication delivery device 102. For example, the information may be indicative of previous events occurring between the medication delivery device and a mobile device (e.g., any mobile device). As described above, an event refers to an occurrence of a mobile device being placed in proximity to the medication delivery device 102 such that data is transmitted from the medication delivery device to the mobile device. The information may include geographical locations 302j associated with the previous event(s) and/or a time(s) of the previous of event(s) (not shown). Additionally or alternatively, the information may indicate an NFC tag identifier 302n associated with the NFC tag 104 disposed on the medication delivery device 102.

A user may have access to at least a portion (e.g., some, most, or all) of the information displayed on website 300. As described herein, different users may have access to different portions of the information. For example, the user may provide user credentials to gain access to user-specific information.

It should be appreciated that the information 302a-n is example information and that any suitable information may be obtained and/or displayed, as aspects of the technology described herein are not limited in this respect.

FIG. 4A is an example display 400 on a user interface of a mobile device showing verification data associated with the medication delivery device, according to some embodiments. The example display 400 shows that the product has been verified. The display 400 also provides information related to the product (e.g., medication and the medication delivery device), such as the manufacturing lot number, the expiration date, the serial number, the manufacturer, and the dose size.

FIG. 4B is an example display 440 on a user interface of a mobile device showing verification data associated with the medication delivery device, according to some embodiments. The example display 440 shows, via graphics and text, that the product has not been verified. This may indicate that the product is a counterfeit product that was not manufactured by the expected, true manufacturer. The example display 440 also prompts the user to report the product, which enables counterfeit tracking and may help to identify the source of the counterfeit products.

According to some embodiments, in addition to an alert shown on the user interface of a patient's mobile device (e.g., as shown in FIG. 4B), an alert may also be sent to the patient's healthcare provider and/or the patient's assistant (e.g., a family member) when a counterfeit product is detected. For example, the healthcare provider and/or assistant may receive a notification via a text message, via a notification on a mobile application, or via any other suitable means of communication, as aspects of the technology are not limited in this respect. Accordingly, the healthcare provider can follow up with the patient to ensure that they do not use the counterfeit product, track the source of the counterfeit product, replace the medication with a verified product to ensure the patient adheres to his or her dosing regimen, and check in with other patients to make sure that they have not received counterfeit products, as well. Similarly, in response to receiving the alert, the patient's assistant can ensure that the patient does not take the counterfeit medication and can order replacement medication to make sure that the patient continues to adhere to the dosing regimen.

FIG. 4C is an example display 460 on a user interface of a mobile device showing a recall notice associated with the medication contained within the medication delivery device, according to some embodiments. A warning message at the top of the screen indicates that the product (e.g., the medication delivery device and the medication contained within the medication delivery device has been recalled.) Also shown in the bottom of display 460, is an interactive button that, when selected, will provide instructions for how to use the medication delivery device.

FIG. 4D is an example display 480 on a user interface of a mobile device showing information indicating an expiration of a medication contained within the medication delivery device, according to some embodiments. A warning message at the top of the screen indicates that the medication is expired and provides instructions for the user to tap the message for additional information. Again, this warning message instructs the user to tap the message for additional details regarding the recall notice.

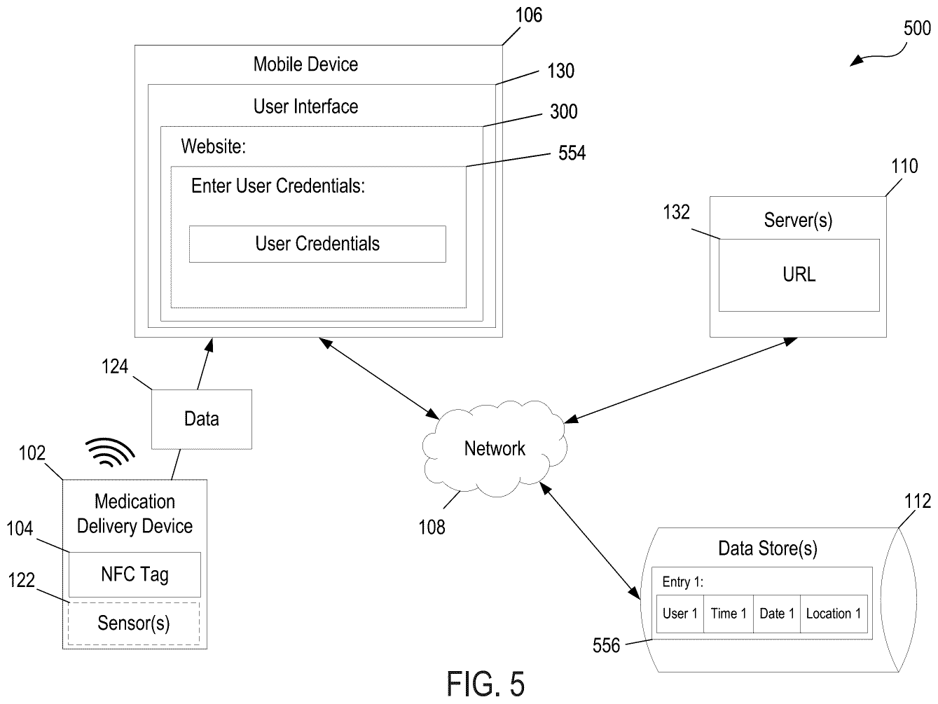

FIG. 5 is a block diagram depicting an example system 500 for tracking possession of a medication delivery device, according to some embodiments. Like system 100, system 500 includes medication delivery device 102 and mobile device 106, which communicates with server(s) 110 and data store(s) 112 via network 108.

However, unlike system 100, system 500 is additionally or alternatively configured to track possession of the medication delivery device 102. In some embodiments, the mobile device 106 of system 500 is configured to receive data 124 from the medication delivery device 102, as described herein with respect to FIG. 1. After receiving the data (e.g., in response to receiving the data), the mobile device launches a web browser configured to access a URL 132 associated with server(s) 110. Additionally or alternatively, the mobile device 106 checks for and launches a previously downloaded application program (e.g., a mobile app) configured to access the URL 132 associated with the server(s) 110.

The mobile device 106 transmits at least a portion (e.g., some, most, or all) of the data 124 to the server(s) 110. In some embodiments, the portion of data 124 includes data indicative of the authentication code generated by the NFC tag 104 and/or data indicative of the output of sensor(s) 122. In some embodiments, the mobile device 106 also transmits, to server(s) 110, contextual information, such as a time, date, and/or geographical location of the mobile device 106 when the data 124 was received from the NFC tag 104 disposed on the medication delivery device 102.

In response, a website 300 is displayed on the user interface 130 of the mobile device. The website 300 is configured to prompt a user for user credentials 554. For example, the user may provide a username and password via one or more text boxes presented on user interface 130. Additionally or alternatively, the user credentials may include a bearer token, a refresh token (e.g., derived from previous authentication using a username and password), and/or other user credentials.

Mobile device 106 transmits user credentials to the server(s) 110. Server(s) 110 are configured to log an entry 556 in data store(s) 112 based on the user credentials 554 and the portion of data 124 transmitted by the mobile device 106. The entry 556 may indicate the user (e.g., the user who provided the user credentials 554), the time at which the data 124 from the medication delivery device 102 was received by the mobile device 106, the date on which the data 124 was received by the mobile device 106, and/or the geographical location of the mobile device 106 when it received the data 124 from the medication delivery device 102. It should be appreciated, however, that the entry 556 may include less information, different information, and/or additional information, as aspects of the technology are not limited in this respect.

In some embodiments, the logged entry 556 may be accessed by one or more users to track patient adherence, detect potential counterfeiting, determine a time for a next dose, gather data from a clinical trial, track device distribution, and/or any other suitable use case, as aspects of the technology described herein are not limited in this respect. For example, users may access the information through a website configured to access this data.

In some embodiments, server(s) 110 may also look up the received user credentials 554 in data store(s) 112 to identify additional, user-specific information associated with the user credentials 554. For example, the server(s) 110 may access a dosing regimen specific to the user, past doses administered to the user, patient information associated with the user, or any other suitable user-specific information, as aspects of the technology described herein are not limited in this respect.

Server(s) 110 may provide the patient specific information and/or other information described herein including the information described with respect to FIGS. 1-4D to mobile device 106. Mobile device 106 may display the information on a website displayed by a web browser and/or on application program executing on the mobile device 106. In some embodiments, certain information may only be displayed to some users (e.g., based on user credentials 554), while the same information is not displayed to other users.

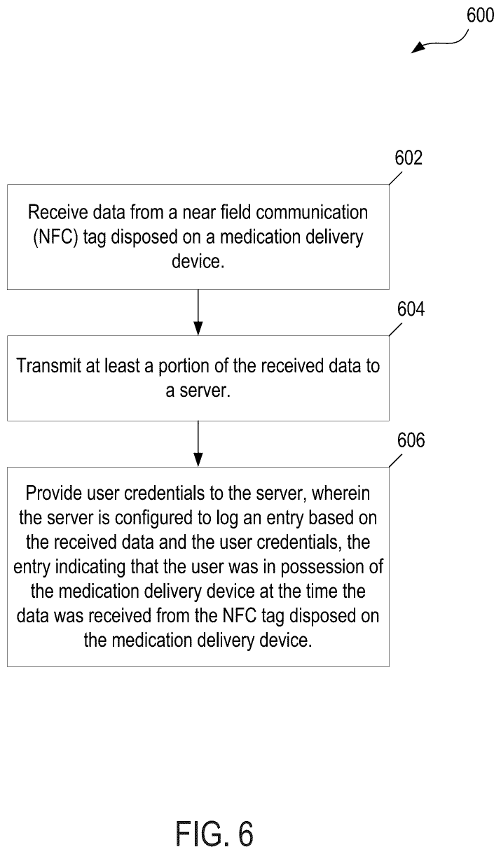

FIG. 6 is a flowchart showing an exemplary computerized method 600 for tracking possession of a medication delivery device, according to some embodiments. Method 600 may be implemented on any of one or more of mobile device 106 and server(s) 110, whether working independently or in cooperation with one another.

At step 602, the mobile device receives data from a near field communication (NFC) tag disposed on a medication delivery device. The NFC tag may include an NFC chip and one or more NFC antennas disposed on the medication delivery device. For example, the NFC tag may include an NFC chip and one or more NFC antennas incorporated into a label of the medication delivery device. The label may be affixed to, adhered to, or formed as part of the medication delivery device. An exemplary label, which incorporates an NFC tag, is described herein including at least with respect to FIGS. 15A and 15B.

In some embodiments, the mobile device receives the data in response to coming within proximity to the medication delivery device. This may include coming within 10 cm, within 8 cm, within 6 cm, within 4 cm, within 2 cm, between 0 and 10 cm, between 2 and 8 cm, or within any suitable distance of the medication delivery device. For example, a user can tap the medication delivery device against the mobile device (or vice versa), and, as a result, the mobile device receives data from the medication delivery device. The mobile device may receive the data via a wireless signal received by a communication circuit of the mobile device. The wireless signal may be an active signal or a passive signal.

The received data may include data associated with the NFC tag disposed on the medication delivery device and/or data associated with one or more sensors associated with the medication delivery device. As described herein, including at least with respect to FIG. 1, data associated with the NFC tag may include data indicative of an authentication code generated by the NFC tag. In some embodiments, the data indicative of the authentication code is encrypted data. In some embodiments, the data associated with the one or more sensors includes data indicative of the output of the one or more sensors, such as the sensors described herein including at least with respect to FIG. 1.

According to some embodiments, in response to receiving data from the NFC chip, the mobile device determines information indicative of an event. Such information may include contextual information such as the time, date, and/or geographical location of the mobile device when it received data from the medication delivery device. According to some embodiments, the mobile device determines the contextual information based on the on-board clock, calendar, and/or GPS chip at the time the data was received from the NFC tag.

At step 604, the mobile device transmits at least a portion of the received data to a server. For example, the mobile device may transmit some, most, or all of the received data to the server via any suitable network. In some embodiments, the mobile device also transmits a portion of the contextual information to the server, such as the time, date, and/or geographical location of the mobile device when it received the data from the medication delivery device.

In some embodiments, the server is configured to, in response to receiving at least a portion of the data from the mobile device, launch a website on the mobile device. The website is configured to prompt a user to provide user credentials using a user interface of the mobile device. For example, a user may provide a username and password using one or more text boxes displayed on the user interface. Additionally or alternatively, the user credentials may include a bearer token, a refresh token, and/or other user credentials, as described herein.

At step 606, the mobile device provides the user credentials to the server. The server is configured to log an entry based on the user credentials and the portion of the received data (e.g., transmitted to the server at step 602). For example, the server may be configured to log the entry in a data store associated with the server. The entry indicates that the user was in possession of the medication delivery device at the time the data was received from the NFC tag disposed on the medication delivery device. For example, the entry may indicate the user credentials, a user associated with the user credentials, a geographical location, a time, a date, and/or any other suitable information, as aspects of the technology are not limited in this respect.

### Example Applications

Described herein are exemplary applications of the systems and methods for verifying an aspect of a medication delivery device and tracking possession of a medication delivery device, as described above including at least with respect to FIGS. 1-6. However it should be appreciated that the following examples are nonlimiting examples, and the systems and methods described herein may have many other suitable applications.

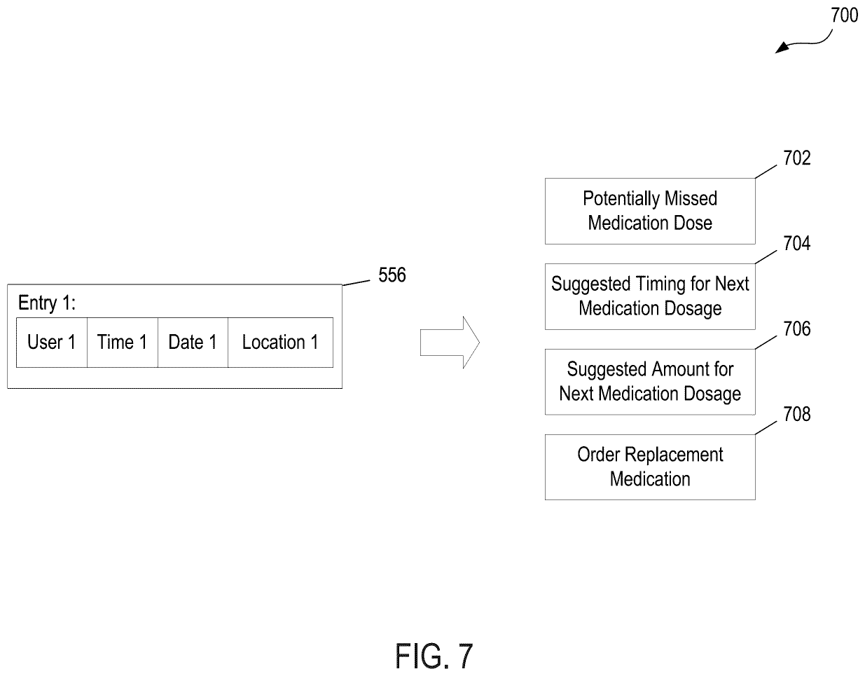

FIG. 7 is a block diagram depicting an example system 700 for using a logged entry to determine example recommendations associated with the medication delivery device, according to some embodiments.

In this example, a server (e.g., server(s) 110) may access a stored entry 556 that indicates when a patient (e.g., User 1) was administered a dose of medication (e.g., an event). The server may also access additional information stored with entry 556 that is related to the event and/or the patient. For example, the entry 556 may store data indicative of the dose of medication that was delivered, the name of the medication, the patient's dosing regimen, information related to the timing of other past dosing events, and/or any other suitable information.

In some embodiments, the server uses the entry 556 and/or information stored with the entry 556 to track when the patient took or received the medication. With that information, the server can provide assistance to the patient or the patient's caregiver (e.g., healthcare provider, family member, etc.). For example, the server can detect a potentially missed medication dose 702. The server can detect potentially missed medication doses if no data is received from the mobile device during a time window when the patient would be expected to have taken a medication dose. The server can also suggest a time for the next medication dose 704, suggest a dose amount for the next medication dose 706, and/or order replacement medication 708.

The server may transmit data related to these options 702-708 to the mobile device, and information indicative of the data may be displayed on a website on the user interface of the mobile device. For example, the website may provide messages to the patient, such as messages warning the patient that they may have missed a dose, suggesting the time for the next dose, suggesting the amount for the next dose, and/or indicating that replacement medication has been ordered.

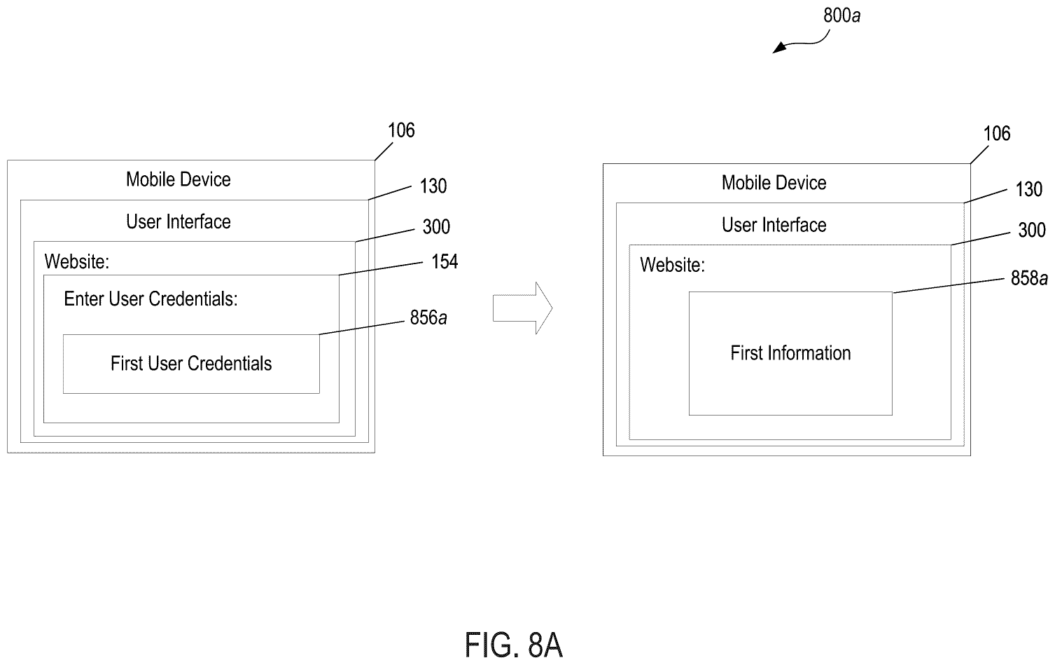

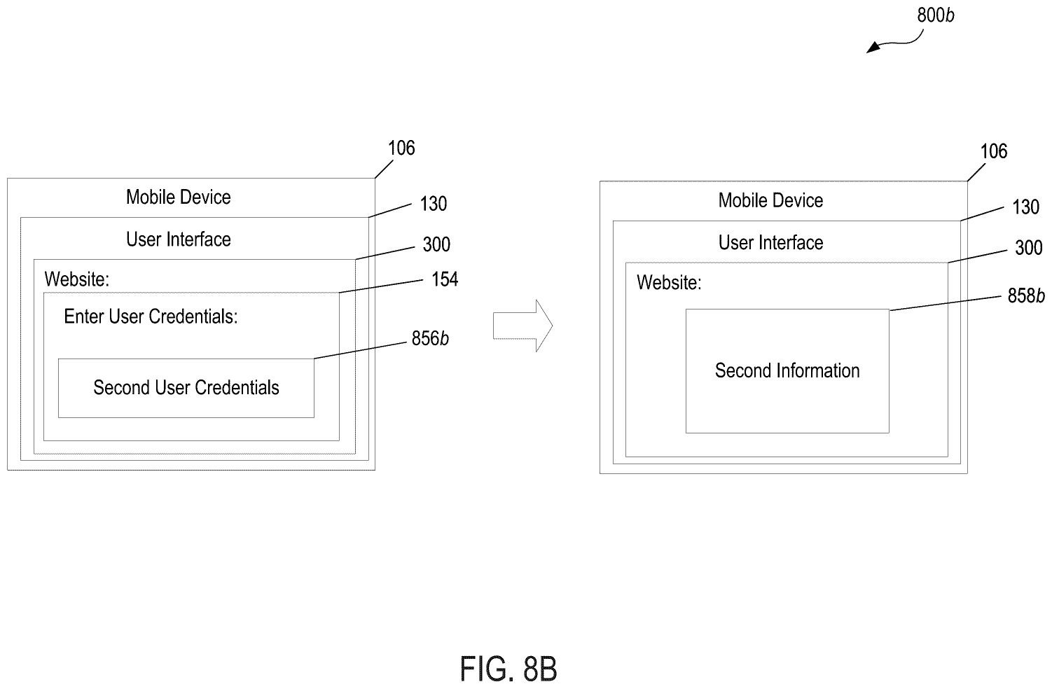

It should be appreciated that by providing user credentials, the user can access patient-specific information that allows the user to keep track of his or her dose regimen, receive communications from their healthcare provider, and maintain their prescriptions. As shown in FIGS. 8A and 8B, a first user may provide first user credentials 856a that are unique to the first user, while a second user may provide second user credentials 856b that are unique to the second user.

In response to receiving the user credentials, the server is configured to provide user-specific data to the mobile device 106. Accordingly, the website 300 displays first information 858a that is specific to the first user when the first user credentials 856a are provided, and second information 858b that is specific to the second user when the second user credentials 856b are provided.

A research investigation and/or a clinical trial may seek to determine how well a group of patients complies with therapy. Relatedly, an insurance company may seek to track population-level health outcomes. By automatically logging entries associated with dosing events, the techniques described herein can be used to determine a level of adherence for the group of patients.

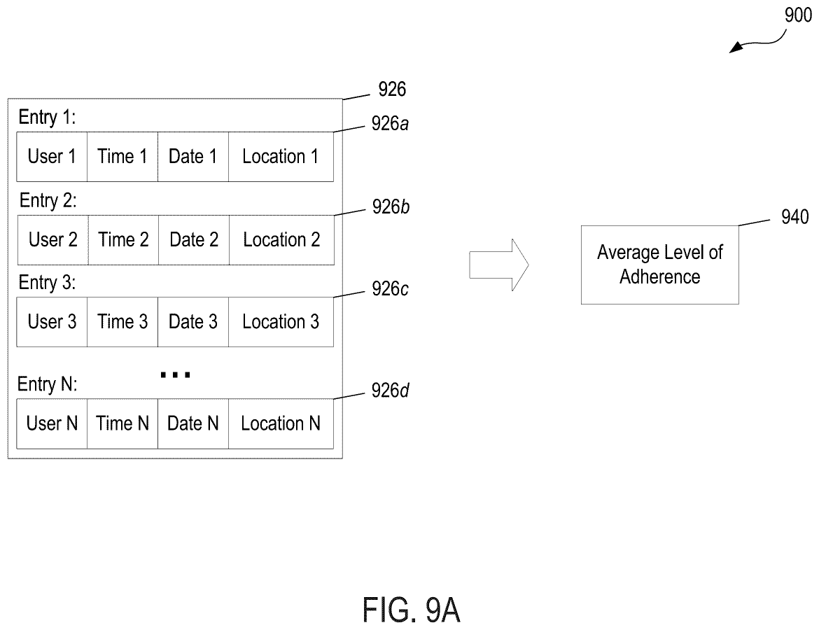

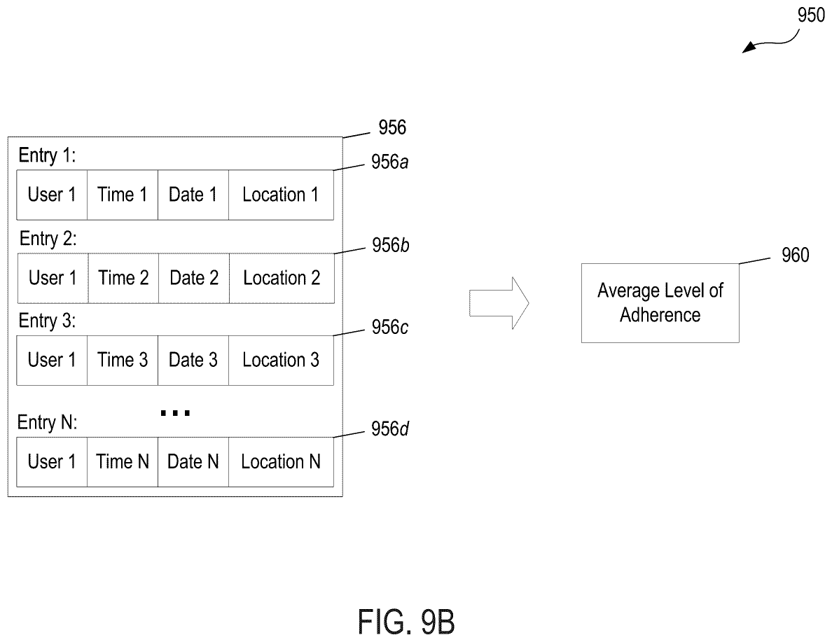

For example, as shown in FIG. 9A, a server and/or a user (e.g., a researcher and/or insurance company) may access logged entries 926 indicating dosing events for multiple patients (e.g., users 1-N). The server and/or user may use the logged entries 926 to determine the average level of adherence 940 of those patients to their dosing regimens. Additionally or alternatively, the user and/or server may access individual entries 926a-d to evaluate the adherence of individual patients to their individual dosing regimens.

In some embodiments, a healthcare provider may seek to determine how well an individual patient complies with therapy. In particular, healthcare providers may wish to track when a patient takes his or her medication and whether the patient is adhering to his or her medication regimen. By automatically logging entries associated with dosing events, the techniques described herein can be used to provide patient adherence information to healthcare providers.

For example, as shown in FIG. 9B, a server and/or a user (e.g., a healthcare provider) may access logged entries 956 indicating dosing events for a single patient (e.g., user 1). The server and/or user may use the logged entries 956 to determine the average level of adherence 960 of the patient to his or her dosing regimen. Additionally or alternatively, the user and/or server may access individual entries 956a-d to evaluate the timing and/or dosage amounts administered to the user.

Accordingly, healthcare providers can provide better care for their patients by identifying and addressing issues that arise when a patient is not adhering to his or her medication regimen and developing effective therapy plans.

Sales representatives that distribute medication samples (e.g., contained within medication delivery devices) to healthcare provider offices are under regulatory obligation to keep track of which medication samples they have distributed to whom. The sales representative typically logs information about the sample, such as the medication product, manufacturing lot number, date of visit, doctor's office provided to, etc. This process is manual and time consuming.

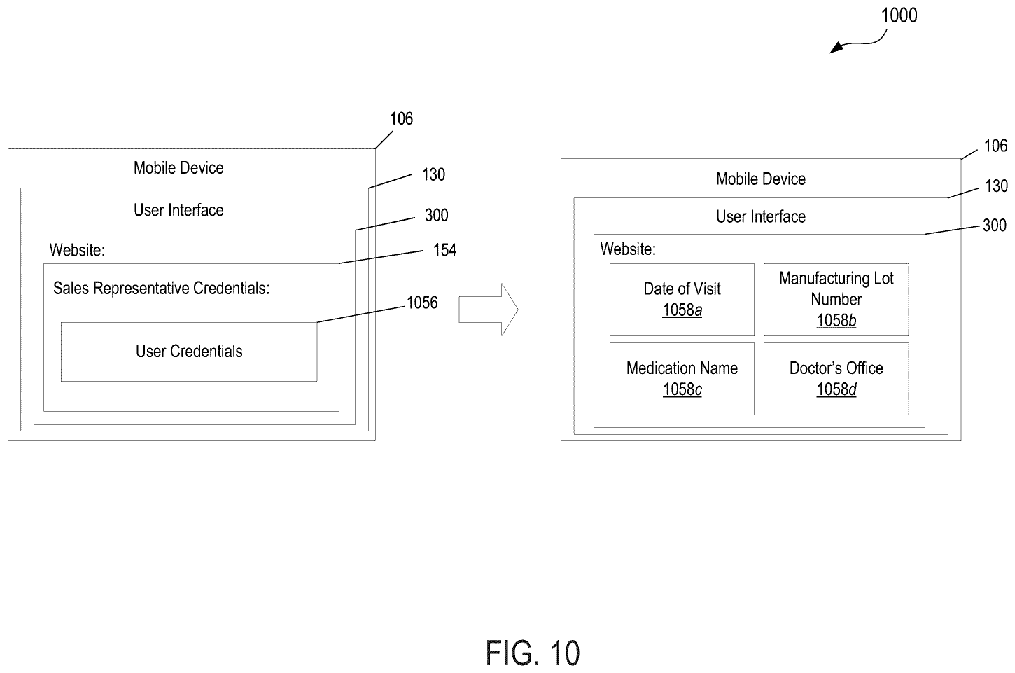

The efficiency of this process can be improved upon using the techniques described herein. As shown in FIG. 10, the sales representative may tap the medication delivery device to a mobile device 106. The website 300 prompts the sales representative for credentials 1056, and, upon providing the credentials, the system logs the information. For example, the system logs the date of visit 1058a, manufacturing lot number 1058b, medication name 1058c, and doctor's office 1058d. However, it should be appreciated that any suitable information may be logged, as aspects of the technology are not limited in this respect.

The sales representative may access the logged information at a later time by providing his or her credentials 1056, and the website 300 will display the logged information via a user interface 130 on the mobile device 106.

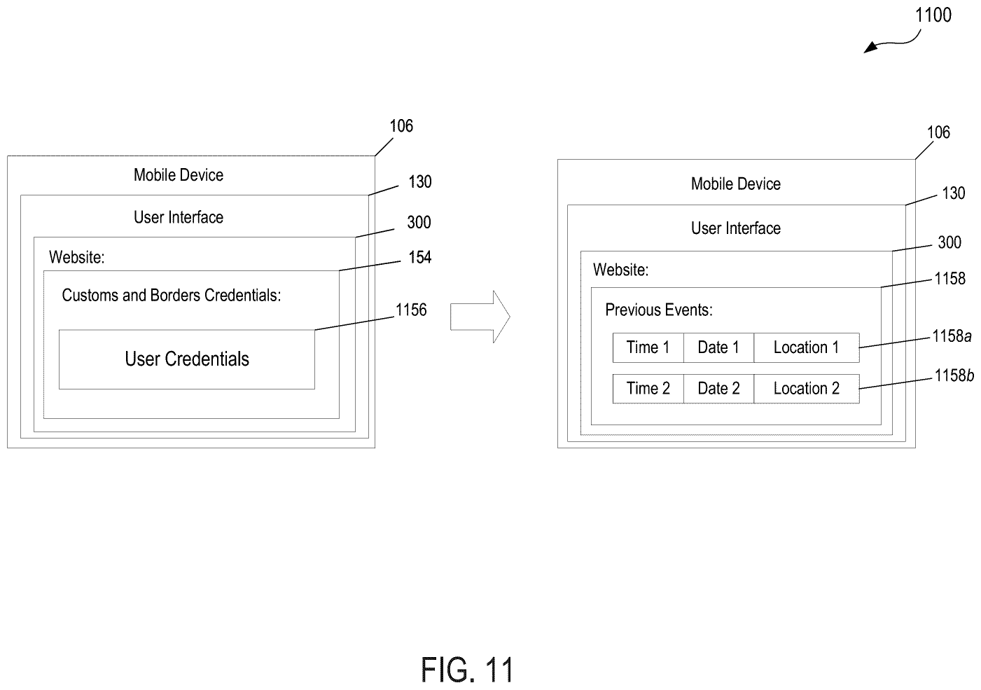

In some embodiments, a customs and borders agent may check a medication delivery device to determine whether it is a counterfeit. FIG. 11 is a block diagram depicting an example system 1100 for detecting a counterfeit product, according to some embodiments of the technology.

The agent may tap the medication delivery device to a mobile device 106, which launches website 300 that prompts the agent for their credentials 1156. After credentials are provided, the website 300 may display information about previous events 1158. For example, the information may include previous geo-locations 1158a and 1158b (e.g., determined from IP addresses) at which the NFC tag was previously tapped. Such information may not be generally available to the public.

The customs and borders agent may use the geo-location information to determine the likelihood that the medication delivery device is a counterfeit. For example, if the information indicates that the NFC tag was previously tapped in another continent and that only a short period of time had elapsed since the NFC tag was tapped, then it is likely that the medication delivery device is a counterfeit.

In some embodiments, the customs and borders agent may provide information to website 300, such as information reporting a counterfeit product. This information can be used to track down sources of counterfeit goods.

In clinical trials, researchers often provide the drug to be tested to a first subset of a patient population and a placebo to a second subset of the patient population. This allows researchers to measure the effectiveness of the drug in treating a condition relative to a placebo. In order to prevent potential bias, researchers “double-blind” the distribution of the drug and the placebo. That is, neither the patients, nor the healthcare provider distributing the product to the patients, know whether the product being administered to a particular patient is a drug or a placebo. In such double-blinded drug trials, all the external labels for the distributed drug containers may look the same, regardless of whether the container contains a drug or a placebo.

Despite identical, external container labelling, an NFC tag may be incorporated into the label of each drug container, allowing patients to retain individual serialization of the drug container. By tapping a mobile device to the drug container before administering the product (e.g., drug or placebo), an entry is logged that stores the patient's information and information identifying the product contained within the container. Therefore, researchers on the back-end can track which patients took the drug being tested and which patients took the placebo. That is, once the data from the clinical trial is unblinded, researchers can determine which patients took the placebo versus which patients took the active ingredient based on the logged entries that store the patient's information and information identifying the product contained within the container.

Additionally or alternatively, researchers can easily communicate with patients participating in the clinical trial by updating the website accessed by the patients. That is, when a patient taps the drug container to a mobile device (or vice versa), a website is launched that displays information indicating updates to the clinical trial.

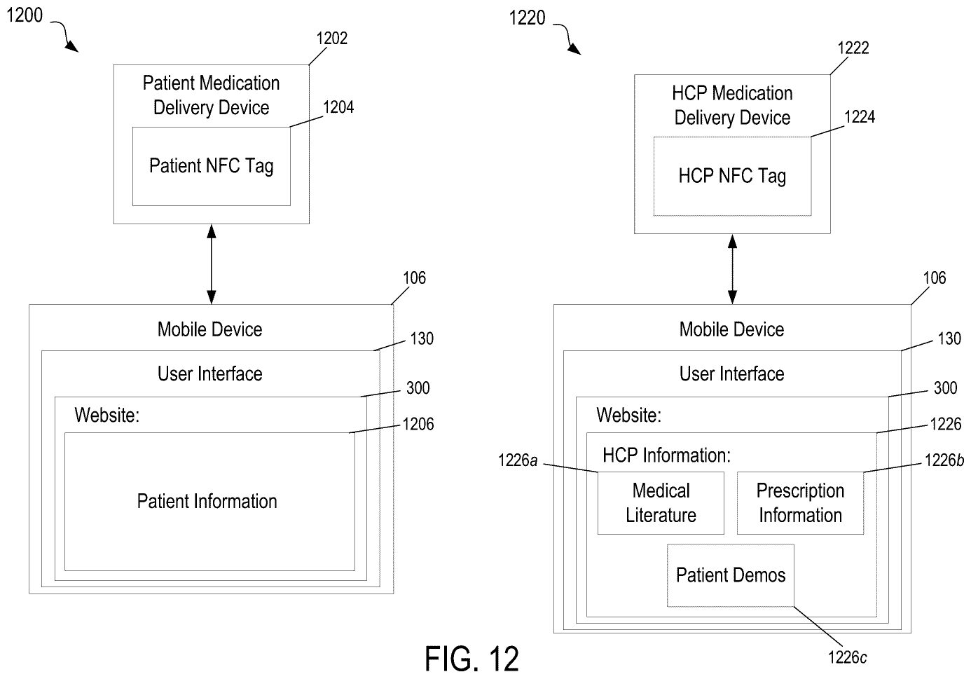

According to some embodiments, a medication delivery device may be configured to be used by a patient. For example, as shown in FIG. 12, the patient medication delivery device 1202 of example system 1200 includes a patient NFC tag 1204. When the mobile device 106 is placed in proximity to the patient medication delivery device 1202, a website 300 is launched on the user interface 130 of the mobile device 106.

According to some embodiments, in response to receiving data from the patient NFC tag 1204, the website 300 displays patient information 1206. Patient information 1206 may include, for example, information about the patient medication delivery device, information about the medication contained within the patient medication delivery device, instructions for how to use the patient medication delivery device, a dosing schedule for the patient, medical history information for the patient, or any other suitable general or patient-specific information, such as the information described herein including at least with respect to FIG. 3.

Additionally or alternatively, a medication delivery device may be configured to be used by a healthcare provider (HCP). For example, as shown in FIG. 12, the HCP medication delivery device 1222 of example system 1220 includes an HCP NFC tag 1224. When the mobile device 106 is placed in proximity to the HCP medication delivery device 1222, a website 300 is launched on the user interface 130 of the mobile device 106.

According to some embodiments, in response to receiving data from the HCP NFC tag 1224, the website 300 displays HCP information 1226. HCP information 1226 may include, for example, medical literature 1226a, prescription information 1226b, patient demos 1226c, or any other suitable general or HCP-specific information, such as the information described herein including at least with respect to FIG. 3. In some embodiments, the prescription information 1226b includes information that describes how and when to prescribe to patients. In some embodiments, the patient demos 1226c include features that allow an HCP to demonstrate to patients how something is to be done medically or electronically. For example, the patient demos 1226c may include features that allow an HCP to demonstrate to patients how to sign Health Insurance Portability and Accountability Act (HIPAA) forms electronically. Alternatively or additionally, the patient demos 1226c may facilitate the HCP and the patient to locate and actually electronically sign the appropriate HIPAA forms. The electronically signed forms may then be stored in the appropriate electronic repositories, such as an Electronic Health Record (EHR) system.

Patients sometimes appreciate receiving reminders to take their medications. For example, some medications need to be stored in a refrigerator to prevent spoliation. When it is time to take a dose of medication, patients are instructed to take the medication (e.g., the medication delivery device) out of the refrigerator and to let it rest at room temperature for a specified duration of time (e.g., 15 minutes, 20 minutes, 30 minutes, 40 minutes, 50 minutes, 60 minutes, or any other suitable duration of time). Some patients would appreciate receiving a reminder after the specified duration of time that their medication is now ready to be taken. Additionally or alternatively, some patients would appreciate general reminders to take their medication according to their medication regimen (e.g., multiple times per day, daily, weekly, etc.).

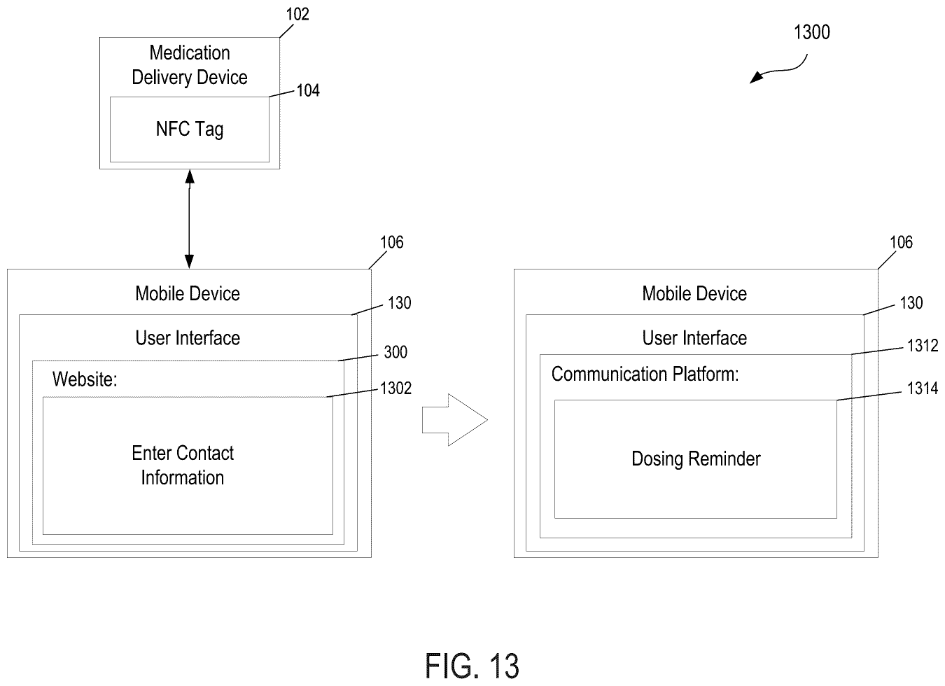

FIG. 13 shows an example system 1300 for configuring dosing reminders, according to the techniques described herein. As described herein, according to some embodiments, after mobile device 106 is placed in proximity to the medication delivery device 102, a website 300 is launched on the user interface 130 of the mobile device 106.

According to some embodiments, the website 300 is configured to prompt a user to enter contact information 1302 for configuring dosing reminders. For example, the website 300 may display options for configuring dosing reminders 1314 via one or more communication platforms 1312. A user may then enter his or her contact information 1302 for the respective communication platform 1312. For example, the user may provide a phone number to receive dosing reminders via short message service (SMS) text, an email address to receive dosing reminders via email, a username to receive dosing reminders via a messaging platform, or any suitable contact information for any suitable communication platform, as aspects of the technology described herein are not limited in this respect.

After the user provides contact information, according to some embodiments, a server (e.g., server(s) 110) provides the user with a code via the communication platform 1312. For example, if the user provided a phone number, he or she may receive the code via text message. The website 300 may prompt the user to enter the code via the user interface 130 to confirm that the patient is in possession of the communication platform 1312 (e.g., email account, phone number, messaging platform) associated with the provided contact information.

According to some embodiments, after the dosing reminders have been configured, the user receives the dosing reminders 1314 via the communication platform 1312. The dosing reminders 1314 remind the user to take his or her medication at the appropriate time. For example, the user may receive a dosing reminder after a specified duration of time has elapsed (e.g., 30 minutes) since the medication was removed from a refrigerator. In this case, the dosing reminder may indicate that the medication has warmed up and is ready for use. Additionally or alternatively, the user may receive dosing reminders at certain times each day or at certain times of the week. Such a dosing reminder may help a user adhere to his or her medication regimen.

Additionally or alternatively, when setting up dose reminders for the first time via website 300, or upon receiving a dose reminder via communication platform 1312, the user may be provided with an option to input an injection site that the user used in his/her most recent injection. For example, when setting up dose reminders for the first time via website 300, the website may include user-entry fields allowing a user to choose between multiple options for injection sites, such as left arm, right arm, left abdomen, right abdomen, left thigh, and/or right thigh. Additionally or alternatively, the website 300 may allow a user to type free text indicating the injection site that the user used in his/her most recent injection. When receiving a dosing reminder via communication platform 1312, the dosing reminder may include similar user-entry fields, or the dosing reminder may include a link to a website comprising similar user-entry fields. When the user provides information regarding the injection site that the user used in his/her most recent injection, this information may be saved by a server (e.g., server(s) 110) in a log of previously-used injection sites, potentially in association with other injection-related data, such as the date and/or time of the previous injection associated with a previous injection site, type of medication injected, and/or amount of medication injected. This logged information may be accessed by the user upon request, or presented to the user along with each medication reminder, to remind the user regarding which injection sites he/she had used in his/her most recent injections. In this way, the logged information may assist the user to rotate injection sites effectively and not over-use any particular injection site, as over-using a particular injection site may increase the risk of complications (e.g., infection or scarring) and/or decrease the effectiveness of the injected medication.

An exemplary medication delivery device 20 is illustrated in various operational states in FIGS. 14A-14C. Examples of such a device and its operation are described in U.S. Pat. No. 8,734,394 B2 issued May 27, 2014 to Adams et al. and in U.S. Patent App. Pub. No. 2021/0093784 A1 published Apr. 1, 2021 to Adams et al., the entire disclosure of each of which is hereby incorporated herein by reference. Device 20 includes a syringe assembly 22, a drive mechanism 24, and a retraction mechanism 26. Syringe assembly 22 includes a barrel 30 forming a reservoir for holding a medication, and a piston 32 disposed within the barrel 30 for driving the medication outside the barrel. Syringe assembly 22 also includes a needle assembly 33 having a hollow injection needle 34 and a needle hub 35 which mounts needle 34 to syringe barrel 30. A lower body support member 29 coupled to device housing 38 surrounds needle 34. Advancing piston 32 within barrel 30 toward needle 34 dispenses medication through needle 34.

Devices described herein, such as device 20, may further comprise a medication, such as for example, within the syringe barrel 30. In another embodiment, a system may comprise one or more devices including device 20 and a medication. The term “medication” or “drug” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, combined GIP/GLP-1 agonists such as tirzepatide, oxyntomodulin analogs, oxyntomodulin derivatives, therapeutic antibodies including but not limited to IL-23 antibody analogs or derivatives, such as mirikizumab, IL-17 antibody analogs or derivatives, such as ixekizumab, therapeutic agents for pain-related treatments, such as galcanzeumab or lasmiditan, and any therapeutic agent that is capable of delivery by the devices described herein. The medication as used in the device may be formulated with one or more excipients. The device is operated in a manner generally as described above by a user, caregiver or healthcare professional to deliver medication to a patient. As used herein, the term “user” may refer to an operator of the devices described herein, and the term “patient” may refer to a person receiving the medication. In some cases, the user and the patient may be the same person (e.g., the patient is operating the devices described herein to give him/herself an injection). In other cases, the user and the patient may be different persons (e.g., the user may be a person providing care to the patient).

FIG. 14A illustrates device 20 in its initial, pre-use configuration. Here, an end cap 36 is secured to lower body support member 29 (which is in turn coupled to device housing 38). End cap 36 covers a proximal end opening 40 in housing 38. As used herein, distal and proximal refer to axial locations relative to an injection site when the apparatus is oriented for use at such site, whereby, for example, proximal end of the housing refers to the housing end that is closest to such injection site, and distal end of the housing refers to the housing end that is farthest from such injection site. Also as used herein, an “injection site” may refer to the exact spot on a patient's body that is injected by a needle, as well as body tissue surrounding the spot where the needle injects (e.g., within 1-5 cm or 1-10 cm of the spot where the needle punctures the patient's skin). Housing 38 may be formed from a plastic material and is shown extending generally longitudinally between a distal end in close proximity to an actuating button 52 and a proximal end in close proximity to the proximal end opening 40 along a longitudinal axis 48. As shown in FIG. 14B, housing 38 may comprise a user-graspable portion 37 configured to be grasped by a hand of a user, the user-graspable portion 37 extending a radial distance 41 outward from longitudinal axis 48. In some embodiments, the radial distance 41 may be between 5-10 mm in length (e.g., in some embodiments, 5-8 mm may be a suitable length). Also as shown in FIG. 14B, housing 38 may also comprise an outwardly-flared end portion 39 at a proximal end of the housing adjacent the proximal opening 40.

A needle guard 42 is mounted on syringe assembly 22 and covers and surrounds needle 34. End cap 36 and needle guard 42 protect the user from accidental needle pricks and also protect needle 34 from damage. When using device 20 to dispense medication, for example, injecting the medication into a patient, end cap 36 and needle guard 42 are first removed. FIG. 14B illustrates device 20 after removal of end cap 36 and needle guard 42 from syringe assembly 22, wherein the syringe assembly is in a storage position and device 20 is ready for a dispensing event.

Syringe assembly 22 is moveable relative to the medication-delivery device 20 between a storage position and an injection position. FIG. 14C illustrates device 20 after the syringe assembly 22 has been moved relative to device 20 to an injection position from its storage position that is shown in FIG. 14B. In the storage position (FIGS. 14A and 14B), needle 34 is retracted to a position such that needle 34 is disposed within housing 38 of device 20. In the injection position (FIG. 14C), needle 34 projects outwardly from housing 38 beyond proximal opening 40 in the proximal direction parallel to longitudinal axis 48 whereby needle 34 may be inserted into a patient.

Drive mechanism 24 includes a plunger 44 which engages piston 32. Drive mechanism 24 includes a spring 46 that drives plunger 44 in a translational movement. In the illustrated embodiment, spring 46 advances plunger 44 along a linear path defined by the longitudinal axis 48 of device 20. As plunger 44 is advanced, foot 50 of plunger 44 contacts piston 32. As the plunger 44 is further advanced, syringe assembly 22 is advanced along axis 48 from its storage position to its injection position. After advancement of syringe assembly 22 to its injection position, the continued proximal advancement of plunger 44 advances piston 32 proximally within barrel 30 from its initial piston position (shown in FIGS. 14A and 14B) to its final piston position (shown FIG. 14C) to cause medication to be dispensed from needle 34 in a dispensing event. Prior to any dispensing of medication and when syringe barrel 30 holds the full original volume of medication, piston 32 will be in its initial piston position. After advancing piston 32 the full extent of its travel length toward needle assembly 33, piston 32 will be in its final piston position proximate needle assembly 33 and the medication from within barrel 30 will have been discharged. In some embodiments, syringe assembly 22 will hold a single dose of medication which will be delivered in a single injection event and piston 32 will be advanced from its initial piston position to its final piston position in that single injection event to thereby deliver the entire single dose contents of syringe assembly 22. While the device is shown as a single use device, device 20 may also be configured as a multiple-use device with appropriate modifications.

The advancement of plunger 44 will generally not result in the dispensing of medication from syringe assembly 22 until after syringe assembly 22 has been advanced to the injection position. There are factors that may inhibit the medication from being dispensed before the syringe is advanced to the injection position. A factor may be the friction between piston 32 and barrel 30. Typically, piston 32 will be formed out of a rubber material and barrel 30 will be glass. The frictional resistance between these two components may be sufficient to prevent the advancement of piston 32 within barrel 30 until syringe assembly 22 is advanced to its injection position and engagement with a suitable stop member prevents the further advancement of syringe assembly 22. Additionally, the medication within the syringe may be somewhat viscous and thereby somewhat resistant to flowing out of needle 34. If necessary, modification of piston 32 and syringe barrel 30 to alter the frictional resistance of the dispensing motion of the engagement member 32 relative to syringe barrel 30 may limit or prevent the premature dispensing of medication before container 22 reaches its injection position.

To activate drive mechanism 24, a person depresses actuating button 52 at the distal end of device 20. Depressing button 52 disengages one or two elongate prongs 54 on plunger 44 from a shuttle assembly 60 thereby allowing spring 46 to expand so as to axially advance plunger 44. Spring 46 has a helical shape and surrounds prongs 54. The proximal end of spring 46 biasingly engages a flange on plunger 44.

Shuttle assembly 60 may include an upper shuttle member 62 and a lower shuttle member 64. Shuttle members 62, 64 are fixed together in the final assembly. In the final assembly, upper shuttle member 62 captures button 52 and spring 46 limiting the axial movement of these parts in the distal direction. Prongs 54 engage surfaces on upper shuttle 62 when the device is in the condition shown in FIGS. 14A and 14B. Depressing button 52 causes tabs on button 52 to engage ramps (not shown) on prongs 54 to bias prongs 54 inwardly to disengage prongs 54 from upper shuttle member 62. After prongs 54 have been disengaged, spring 46 exerts a biasing force on a flange on plunger 44 to advance plunger 44 from the position shown in FIG. 14B to the position shown in FIG. 14C as spring 46 transitions from its compressed state to an expanded state. As plunger 44 is advanced, it moves syringe assembly 22 to the injection position and then advances piston 32 to dispense medication as discussed above.

After the dispensing event is complete, retraction mechanism 26 optionally moves syringe assembly 22 from the injection position shown in FIG. 14C back to a retracted position. More specifically, the retraction mechanism is adapted to move the medication container from the injection position to the retracted position in a retraction movement. The retracted position may be similar to the storage position in that the syringe assembly is drawn back into the housing 38 such that needle 34 no longer projects proximally from proximal opening 40 and is disposed entirely within housing 38. In some embodiments, the retracted position may be the same as the storage position. In other embodiments, however, a syringe assembly 22 in the retracted position may be located slightly proximal or distal to a syringe assembly in the storage position. In the illustrated embodiment, the retraction mechanism includes a spring 66, a syringe carrier and a rotary member 70 that acts as a follower. In yet other embodiments, the device 20 may include no retraction mechanism 26 such that the syringe assembly remains in its injection position indefinitely after the medication has been dispensed, until the syringe assembly is manually removed or repositioned by a user.

Plunger 44 may include an outrigger (not shown) which unlocks rotary member 70 as plunger 44 nears the end of its travel in the proximal direction. Rotary member 70 is rotationally secured to lower shuttle member 64 by engagement between a latch and a latching recess in lower shuttle member 64. The outrigger unlocks member 70 by depressing the latch. Spring 66 is torsionally preloaded and has one end engaged with member 70 and an opposite end engaged with shuttle assembly 60. Upon depression of the latch, spring 66 causes member 70 to rotate.

Member 70 is rotatable within housing 38 but is not axially moveable relative to housing 38. Other embodiments may include a member 70 that is also axially movable. The rotation of member 70 serves as a delay mechanism to prevent retraction mechanism 26 from retracting syringe assembly 22 until after the syringe assembly has finished delivering its dose of medication. The speed of rotation of member 70 may be adjusted by adjusting a viscosity of grease disposed on or around surfaces of member 70 that are in contact with housing 38—a more viscous grease results in slower rotation, while a less viscous grease results in faster rotation. A radial flange on rotary member 70 may engage a ledge within housing member 38 to limit the proximal movement of member 70. Spring 66 may also be compressively preloaded such that it is initially in a compressed state. In this compressed state, spring 66 may exert an axial force, torsional force, or both forces on member 70 to bias member 70 proximally to thereby maintain member 70 in an axial position where the radial flange of member 70 engages the interior ledge of housing member 38.

Shuttle assembly 60 may include axially extending channels or ribs that engage corresponding features on housing member 38 that allow shuttle assembly 60 to move axially within housing 38 but which prevent the relative rotation of shuttle assembly 60 relative to housing member 38. Shuttle assembly 60 is biased in the distal direction by spring 66 but is prevented from moving distally by engagement of a latch (not shown) before activation of drive mechanism 24. When rotary member 70 completes its rotation, it disengages the aforementioned latch, thus allowing shuttle assembly 60 to move distally under the biasing force of spring 66.

As shuttle assembly 60 moves distally, it carries syringe assembly 22 distally and moves it back to the retracted position. Also as shuttle assembly 60 moves, spring 66 transitions from its compressed state to an expanded state. Spring 66 biases the retraction mechanism 26 distally and thereby maintains syringe assembly 22 in its retracted position after an injection event. In some embodiments, as shuttle assembly 60 moves distally, spring 46 of drive mechanism 24 may also transition from an expanded state to a compressed state. A locking mechanism such as a detent on the shuttle assembly 60 and a recess on the housing 38 member may additionally provide a locking engagement to secure syringe assembly 22 in the retracted position with needle 34 disposed within housing 38 after an injection event whereby the user may then dispose or otherwise handle device 20 in a safe manner.

FIG. 15A shows an exemplary label 1500 that includes an NFC tag 1504. In some embodiments, the label 1500 is wrapped around an external surface of a medication delivery device. For example, the exemplary label may be wrapped around the external surface of user-graspable portion 37 of medication-delivery device 20. The label 1500 may comprise a flexible paper and/or plastic material that may be attached to the exterior surface of device 20. Label 1500 may be attached using any of a variety of methods, including (but not limited to) using adhesive, adhesion film (e.g., polyurethane films), magnetic attachments, clip-on attachments, ultrasonic bonding/welding, injection molding/in-mold labeling, laser joining/welding, and the like. Label 1500 may be printed with information regarding the device 20 and/or regarding the medication stored within device 20, such as the medication's name, manufacturer name, manufacturing batch/lot number, expiration date, instructions for use and/or storage, the amount of medication stored therein, and the like.

In some embodiments, an NFC tag 1504 is incorporated in label 1500. As described above, a mobile device may be placed in proximity to the NFC tag 1504 to receive data from the NFC tag 1504. The label 1500 may include a graphic 1502 that indicates a position of the NFC tag 1504 within the label 1500. For example, a user may tap a mobile device against the medication delivery device near the graphic 1502 to query the NFC tag 1504.

FIG. 15B shows a view of the back surface of label 1500. As shown, label 1500 comprises the NFC tag 1504. According to some embodiments, the NFC tag 1504 comprises chip 1510 and antenna(s) 1512. According to some embodiments, the NFC tag 1504 may have any suitable dimensions. For example, the NFC tag 1504 may have a width (and/or length) of at least 8 mm, at least 10 mm, at least 12 mm, at least 14 mm, at least 16 mm, at least 18 mm, at least 20 mm, at least 22 mm, at least 24 mm, at least 26 mm, at least 28 mm, at least 30 mm, between 8 mm and 30 mm, between 10 mm and 24 mm, or between 12 mm and 22 mm.

According to some embodiments, the NFC chip 1510 may comprise a processing circuit. Processing circuit 1510 may take the form of a processor (e.g., a microprocessor or microcontroller, field-programmable gate arrays (FPGAs) and/or digital signal processors (DSPs, or any combination of the foregoing) configured to execute logic stored in a memory (not shown) to perform the operations described herein The term “logic”, “control logic”, “instructions” or “application” as used herein may include software and/or firmware executing on any of the aforementioned processing circuits.

In some embodiments, the NFC chip 1510 may be equipped with an on-board temperature sensor and need not be mounted on a flexible substrate that enables the chip to bend. The NFC chip may have any suitable thickness, such as, for example, a thickness of approximately 1 mm or more.

Label 1500 also includes NFC antenna(s) 1512 that are communicably coupled with the NFC chip 1510. In addition to communicating data to an external device, NFC antennas 1512 may also be used to harvest power inductively and wirelessly from said external device.

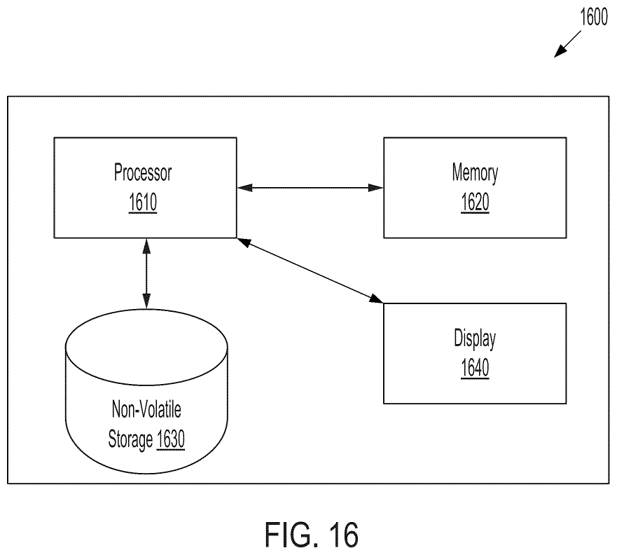

An illustrative implementation of a computer system 1600 that may be used to perform any of the aspects of the techniques and embodiments disclosed herein is shown in FIG. 16. The computer system 1600 may include one or more processors 1610 and one or more non-transitory computer-readable storage media (e.g., memory 1620 and one or more non-volatile storage media 1630) and a display 1640. The processor 1610 may control writing data to and reading data from the memory 1620 and the non-volatile storage device 1630 in any suitable manner, as the aspects of the invention described herein are not limited in this respect. To perform functionality and/or techniques described herein, the processor 1610 may execute one or more instructions stored in one or more computer-readable storage media (e.g., the memory 1620, storage media, etc.), which may serve as non-transitory computer-readable storage media storing instructions for execution by the processor 1610.

In connection with techniques described herein, code used to, for example, identify a patient for inclusion in a clinical trial may be stored on one or more computer-readable storage media of computer system 1600. Processor 1610 may execute any such code to provide any techniques for recognizing objects as described herein. Any other software, programs or instructions described herein may also be stored and executed by computer system 1600. It will be appreciated that computer code may be applied to any aspects of methods and techniques described herein. For example, computer code may be applied to interact with an operating system to recognize objects through conventional operating system processes.

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a suitable framework.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more methods, of which examples have been provided. The acts performed as part of a method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This allows elements to optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting.

Various aspects are described in this disclosure, which include, but are not limited to, the following aspects:

1. A medication delivery device, comprising: a housing comprising a reservoir sized sufficiently to hold medication; an actuating button for initiating an injection of the medication; and a label affixed on at least a portion of the housing, wherein the label comprises a communication tag configured to, when accessed by a mobile device, cause the mobile device to launch a web browser configured to access a uniform resource locator (URL).

2. The medication delivery device of aspect 1, wherein the label comprises at least one of paper and polymer.

3. The medication delivery device of any of aspects 1-2, wherein the tag comprises an antenna and an NFC chip.

4. The medication delivery device of any of aspects 1-3, wherein the URL is associated with an authentication server and comprises an authentication code provided by the tag.

5. The medication delivery device of aspect 4, wherein the authentication server authenticates the authentication code of the URL to authenticate the medication delivery device.

6. The medication delivery device of any of aspects 1-5, wherein the medication delivery device further comprises medication held within the reservoir.

7. The medication delivery device of any of aspects 1-6, wherein the medication delivery device comprises a single-use medication delivery device.

8. The medication delivery device of any of aspects 1-6, wherein the medication delivery device comprises a multi-use medication delivery device.

9. The medication delivery device of aspect 8, wherein the actuating button is rotatable relative to the housing to select a dose size of the medication for injection.

10. A computerized method for verifying an aspect of a medication delivery device, comprising: receiving data from a communication tag disposed on the medication delivery device, wherein the medication delivery device comprises: a housing comprising a reservoir sized sufficiently to hold medication; an actuating button for initiating an injection of the medication; and a label affixed on at least a portion of the housing, wherein the label comprises the tag; verifying the aspect of the medication delivery device by transmitting at least a portion of the received data to a server.

11. The computerized method of aspect 10, wherein the data received from the tag comprises an encrypted code.

12. The computerized method of any of aspects 10-11, wherein verifying the aspect of the medication delivery device comprises verifying a source of the medication delivery device.

13. The computerized method of any of aspects 10-12, further comprising receiving verification data from the server, the verification data being indicative of authenticating the aspect of the medication delivery device.

14. The computerized method of aspect 13, further comprising presenting, via a user interface, information indicative of the verification data.

15. The computerized method of aspect 14, further comprising presenting, via the user interface, additional information associated with at least one of the medication and the medication delivery device.

16. The computerized method of aspect 15, wherein the additional information comprises at least one of a name of the medication, a temperature of the medication, a dose size, a dose strength, an image of the medication delivery device, a unique identifier associated with the tag, an expiration date of the medication, a manufacturing lot number, a recall notice, instructions for administering the medication, a previous geographical location of the medication delivery device, and an activation status of the medication delivery device.

17. The computerized method of any of aspects 14-16, further comprising, upon receiving the data from the tag, launching a web browser configured to access a uniform resource locator (URL), associated with the server, the URL comprising at least one of the information indicative of the verification data and the additional information.

18. The computerized method of any of aspects 15-17, wherein the medication delivery device further comprises a sensor configured to obtain at least some of the information associated with at least one of the medication and the medication delivery device, and wherein the method further comprises receiving the information related to at least one of the medication and the medication delivery device.

19. A non-transitory computer-readable storage media comprising instructions that, when executed by one or more processors on a computing device, are operable to cause the one or more processors to execute the computerized method of any of aspects 10-18.

20. A system comprising a memory storing instructions, and a processor configured to execute the instructions to perform the computerized method of any of aspects 10-18.

21. A computerized method for tracking possession of a medication delivery device, comprising: receiving data from a communication tag disposed on the medication delivery device, wherein the medication delivery device comprises: a housing comprising a reservoir sized sufficiently to hold medication; an actuating button for initiating an injection of the medication; and a label affixed on at least a portion of the housing, wherein the label comprises the tag; transmitting at least a portion of the received data to a server; and providing user credentials to the server, wherein the server is configured to log an entry based on the received data and the user credentials, the entry indicating that the user was in possession of the medication delivery device at a time that the data was received from the tag disposed on the medication delivery device.

22. The computerized method of aspect 21, wherein the entry further indicates a geographical location of the user at the time that the data was received from the tag disposed on the medication delivery device.

23. The computerized method of any of aspects 21-22, further comprising detecting a potential missed medication dose when data is not received from the tag within an expected time.

24. The computerized method of any of aspects 21-22, wherein the server is configured to detect a potential missed medication dose when an entry is not logged within an expected time.

25. The computerized method of any of aspects 21-24, further comprising suggesting at least one of a timing and an amount for a next medication dosage based on the logged entry.

26. The computerized method of any of aspects 21-24, wherein the server is configured to detect at least one of the timing and the next medication dosage based on the logged entry.

27. The computerized method of any of aspects 21-26, further comprising ordering replacement medication or reminding the user to order the replacement medication based on the logged entry,

28. The computerized method of any of aspects 21-26, wherein the server is configured to do at least one of order the replacement medication and remind the user to order the replacement medication based on the logged entry.

29. The computerized method of any of aspects 21-28, wherein the server is configured to log a plurality of entries for a plurality of users and to provide a report on an average level of adherence to a medication regimen among the plurality of users.

30. The computerized method of any of aspects 21-29, further comprising, prior to providing the user credentials to the server, launching a website configured to prompt a user to provide the user credentials.

31. The computerized method of aspect 30, wherein the website is further configured to prompt the user to provide contact information associated with a communication platform, and wherein the computerized method further comprises providing, via the communication platform, a reminder for the user to take a dose of the medication.

32. The computerized method of any of aspects 21-31, further comprising: receiving first user credentials for a first user; and providing first information to the first user based on the first user credentials.

33. The computerized method of aspect 32, further comprising: receiving second user credentials for a second user; and providing second information to the second user based on the second user credentials, wherein the second information is different from the first information.

34. The computerized method of aspect 33, wherein the first information comprises first instructions for using the medication delivery device and the second information comprises second instructions for using the medication delivery device, the first instructions differing from the second instructions.

35. A non-transitory computer-readable storage media comprising instructions that, when executed by one or more processors on a computing device, are operable to cause the one or more processors to execute the computerized method of any of aspects 21-34.

36. A system comprising a memory storing instructions, and a processor configured to execute the instructions to perform the computerized method of any of aspects 21-34.

1. A medication delivery device, comprising:
a housing comprising a reservoir sized sufficiently to hold medication;
an actuating button for initiating an injection of the medication; and
a label affixed on at least a portion of the housing, wherein the label comprises a communication tag configured to, when accessed by a mobile device, cause the mobile device to launch a web browser configured to access a uniform resource locator (URL).

2. The medication delivery device of claim 1, wherein the label comprises at least one of paper and polymer.

3. The medication delivery device of claim 1, wherein the tag comprises an antenna and an NFC chip.

4. The medication delivery device of claim 1, wherein the URL is associated with an authentication server and comprises an authentication code provided by the tag.

5. The medication delivery device of claim 4, wherein the authentication server authenticates the authentication code of the URL to authenticate the medication delivery device.

6. The medication delivery device of claim 1, wherein the medication delivery device further comprises medication held within the reservoir.

7. The medication delivery device of claim 1, wherein the medication delivery device comprises a single-use medication delivery device.

8. The medication delivery device of claim 1, wherein the medication delivery device comprises a multi-use medication delivery device.

9. The medication delivery device of claim 8, wherein the actuating button is rotatable relative to the housing to select a dose size of the medication for injection.

10. A computerized method for verifying an aspect of a medication delivery device, comprising:
receiving data from a communication tag disposed on the medication delivery device, wherein the medication delivery device comprises:
a housing comprising a reservoir sized sufficiently to hold medication;
an actuating button for initiating an injection of the medication; and
a label affixed on at least a portion of the housing, wherein the label comprises the tag;
verifying the aspect of the medication delivery device by transmitting at least a portion of the received data to a server.

11. The computerized method of claim 10, wherein the data received from the tag comprises an encrypted code.

12. The computerized method of claim 10, wherein verifying the aspect of the medication delivery device comprises verifying a source of the medication delivery device.

13. The computerized method of claim 10, further comprising receiving verification data from the server, the verification data being indicative of authenticating the aspect of the medication delivery device.

14. The computerized method of claim 13, further comprising presenting, via a user interface, information indicative of the verification data.

15. The computerized method of claim 14, further comprising presenting, via the user interface, additional information associated with at least one of the medication and the medication delivery device.

16. The computerized method of claim 15, wherein the additional information comprises at least one of a name of the medication, a temperature of the medication, a dose size, a dose strength, an image of the medication delivery device, a unique identifier associated with the tag, an expiration date of the medication, a manufacturing lot number, a recall notice, instructions for administering the medication, a previous geographical location of the medication delivery device, and an activation status of the medication delivery device.

17. The computerized method of claim 14, further comprising, upon receiving the data from the tag, launching a web browser configured to access a uniform resource locator (URL) associated with the server, the URL comprising at least one of the information indicative of the verification data and the additional information.

18. The computerized method of claim 15,
wherein the medication delivery device further comprises a sensor configured to obtain at least some of the information associated with at least one of the medication and the medication delivery device, and
wherein the method further comprises receiving the information related to at least one of the medication and the medication delivery device.

19. A non-transitory computer-readable storage media comprising instructions that, when executed by one or more processors on a computing device, are operable to cause the one or more processors to execute the computerized method of any of claims 10-18.

20-36. (canceled)
